SEQUENCE LISTING (22 sequences)

SEQ ID NO: 1 - protein, 320 aa, Artificial Sequence
  Lip-S1D1-S2D1
  MISC_FEATURE (1)..(1): Lipidation
CSSFNEKGEVSEKIITRADGTRLEYTGIKSDGSGKAKEVLKNFTLEGKVANDKTTLVVKCGTVTLSKNISKSGEVSVELNDTDSSAATKKTAAWNSGTSTLTITVNSKKTKDLVFTKENTITVQQYDSNGTKLEGSAVEITKLDEICNALKGTSDKNNGSGSKEKNKDGKYSFNEKGELSAKTMTRENGTKLEYTEMKSDGTGKAKEVLKNFTLEGKVANDKVTLEVKCGTVTLSKEIAKSGEVTVALNDTNTTQATKKTGAWDSKTSTLTISVNSKKTTQLVFTKQDTITVQKYDSAGTNLEGTAVEIKTLDELCNALK

SEQ ID NO: 2 - protein, 322 aa, Artificial Sequence
  Lip-S4D1-S3hybD1
  MISC_FEATURE (1)..(1): Lipidation
CSSFNAKGELSEKTILRANGTRLEYTEIKSDGTGKAKEVLKDFALEGTLAADKTTLKVTCGTVVLSKHIPNSGEITVELNDSNSTQATKKTGKWDSNTSTLTISVNSKKTKNIVFTKEDTITVQKYDSAGTNLEGNAVEIKTLDELCNALKGTSDKNNGSGSKEKNKDGKYSFNEKGEVSEKILTRSNGTTLEYSQMTDAENATKAVETLKNGIKLPGNLVGGKTKLTVTCGTVTLSKNISKSGEITVALNDTETTPADKKTGEWKSDTSTLTISKNSQKTKQLVFTKENTITVQNYNRAGNALEGSPAEIKDLAELCAALK

SEQ ID NO: 3 - protein, 321 aa, Artificial Sequence
  Lip-S5D1-S6D1
  MISC_FEATURE (1)..(1): Lipidation
CSSFNEKGEISEKTIVRANGTRLEYTDIKSDKTGKAKEVLKDFTLEGTLAADGKTTLKVTCGTVTLSKNISKSGEITVALDDTDSSGNKKSGTWDSGTSTLTISKNRTKTKQLVFTKEDTITVQNYDSAGTNLEGKAVEITTLKELCNALKGTSDKNNGSGSKEKNKDGKYSFNGKGETSEKTIVRANGTRLEYTDIKSDGSGKAKEVLKDFTLEGTLAADGKTTLKVTCGTVVLSKNILKSGEITAALDDSDTTRATKKTGKWDSKTSTLTISVNSQKTKNLVFTKEDTITVQRYDSAGTNLEGKAVEITTLKELCNALK

SEQ ID NO: 4 - DNA, 1020 bp, Artificial Sequence
  Lip-S1D1-S2D1
atgaaagctactaaactggtactgggcgcggtaatcctgggttctactctgctggcaggttgctcaagcttcaacgaaaagggcgaagtcagcgaaaaaatcattacccgcgcagacggcacccgcctggaatacaccggcatcaaatcggacggcagcggcaaagcgaaagaagttctgaaaaactttaccctggaaggcaaagtcgcaaatgataaaaccaccctggtggtgaaatgcggcaccgttacgctgagcaaaaacattagtaaatccggtgaagtctctgtggaactgaatgataccgacagctctgcggccaccaagaaaaccgcagcttggaactcaggcacctcgacgctgaccattacggttaatagcaagaaaaccaaagatctggtcttcacgaaagaaaacaccatcacggtgcagcaatatgacagcaatggtaccaaactggaaggctccgctgtggaaatcacgaaactggatgaaatctgtaatgctctgaaaggtactagtgacaaaaacaatggctctggtagcaaagagaaaaacaaagatggcaagtactcattcaacgaaaaaggcgaactgtcggcgaaaacgatgacgcgtgaaaacggcaccaaactggaatatacggaaatgaaaagcgatggcaccggtaaagcgaaagaagttctgaaaaactttaccctggaaggcaaagtcgccaatgacaaagtcaccctggaagtgaaatgcggcaccgttacgctgtcaaaagaaattgcaaaatcgggtgaagtgaccgttgctctgaacgatacgaataccacgcaagcgaccaagaaaaccggcgcctgggacagcaaaacctctacgctgaccattagtgttaatagcaagaaaaccacgcagctggtcttcaccaaacaagatacgatcaccgtgcagaaatacgacagtgcgggtaccaacctggaaggcacggctgttgaaatcaaaaccctggacgaactgtgtaacgccctgaaa

SEQ ID NO: 5 - DNA, 1029 bp, Artificial Sequence
  Lip-S4D1-S3hybD1
atgaaagctactaaactggtactgggcgcggtaatcctgggttctactctgctggcaggttgctcaagcttcaatgctaagggcgaactgagcgaaaaaacgatcctgcgtgcgaatggcacccgtctggaatacaccgaaatcaaatccgatggtacgggcaaagcaaaggaagtcctgaaagattttgctctggaaggtaccctggcggccgacaaaaccacgctgaaggtgacgtgcggcaccgtggttctgagcaaacatattccgaactctggtgaaatcaccgttgaactgaacgatagcaattctacgcaggcaaccaaaaagacgggcaaatgggacagtaatacctccacgctgaccatttcagtcaactcgaaaaagaccaaaaatattgtgttcacgaaggaagatacgatcaccgttcaaaaatatgactccgcgggcaccaacctggaaggcaatgccgtcgaaatcaaaaccctggatgaactgtgtaacgccctgaagggtactagtgacaaaaacaatggctctggtagcaaagagaaaaacaaagatggcaagtactcattcaacgaaaaaggcgaagtgagcgaaaaaattctgacccgtagcaatggcaccaccctggaatatagccagatgaccgatgcagaaaatgcaaccaaagcagttgaaaccctgaaaaacggtattaaactgcctggtaatctggttggtggtaaaaccaaactgaccgttacctgtggcaccgttaccctgagcaaaaacattagcaaaagcggtgaaattaccgtggcactgaatgataccgaaaccacaccggcagacaaaaaaaccggtgaatggaaaagcgataccagcaccctgaccattagtaaaaatagccagaaaacaaaacagctggtgtttaccaaagaaaacaccattaccgtgcagaattataaccgtgcaggtaatgcactggaaggtagtccggcagaaattaaagatctggcagaactgtgtgcagccctgaaataa

SEQ ID NO: 6 - DNA, 1023 bp, Artificial Sequence
  Lip-S5D1-S6D1
atgaaagctactaaactggtactgggcgcggtaatcctgggttctactctgctggcaggttgctcaagcttcaacgaaaagggcgaaatctcagaaaaaaccatcgtccgcgctaacggcacccgcctggaatacaccgacatcaaatcagacaagaccggtaaagcgaaggaagttctgaaagattttacgctggaaggtaccctggcagcagacggtaaaaccacgctgaaggtgacctgcggtaccgttacgctgtccaaaaacattagtaagtccggcgaaatcacggtcgccctggatgacaccgatagctctggcaacaaaaagagcggtacctgggattcaggcacctcgacgctgaccatttctaaaaatcgtacgaaaaccaagcagctggtcttcacgaaagaagatacgatcaccgtgcaaaactatgacagcgcaggtaccaatctggaaggcaaagctgtggaaattaccacgctgaaagaactgtgtaatgctctgaaaggtactagtgacaaaaacaatggctctggtagcaaagagaaaaacaaagatggcaagtactcattcaacggcaaaggtgaaacgagcgaaaagaccatcgtgcgtgcgaacggtacccgcctggaatatacggacattaaatcggacggcagcggcaaagcaaaggaagtcctgaaagattttacgctggaaggtaccctggcagcagacggtaaaaccacgctgaaggtgacgtgcggcaccgtggttctgtcaaaaaacattctgaagtcgggtgaaatcaccgcagctctggatgacagcgataccacgcgtgctacgaaaaagaccggtaaatgggatagcaagacctctacgctgaccattagtgtcaactcccagaaaacgaagaatctggtgttcaccaaagaagatacgatcaccgttcaacgctatgacagtgcgggcaccaacctggaaggcaaagccgttgaaattaccacgctgaaagaactgtgtaatgctctgaaa

SEQ ID NO: 7 - protein, 958 aa, Artificial Sequence
  C-TAB.G5
MVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTHNNNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYGRSMHNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 8 - protein, 957 aa, Artificial Sequence
  C-TAB.G5.1
VTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYGRSMHNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 9 - DNA, 1557 bp, Artificial Sequence
  C-TAB.G5
atgcataatttgataactggatttgtgactgtaggcgatgataaatactactttaatccaattaatggtggagctgcttcaattggagagacaataattgatgacaaaaattattatttcaaccaaagtggagtgttacaaacaggtgtatttagtacagaagatggatttaaatattttgccccagctaatacacttgatgaaaacctagaaggagaagcaattgattttactggaaaattaattattgacgaaaatatttattattttgatgataattatagaggagctgtagaatggaaagaattagatggtgaaatgcactattttagcccagaaacaggtaaagcttttaaaggtctaaatcaaataggtgattataaatactatttcaattctgatggagttatgcaaaaaggatttgttagtataaatgataataaacactattttgatgattctggtgttatgaaagtaggttacactgaaatagatggcaagcatttctactttgctgaaaacggagaaatgcaaataggagtatttaatacagaagatggatttaaatattttgctcatcataatgaagatttaggaaatgaagaaggtgaagaaatctcatattctggtatattaaatttcaataataaaatttactattttgatgattcatttacagctgtagttggatggaaagatttagaggatggttcaaagtattattttgatgaagatacagcagaagcatatataggtttgtcattaataaatgatggtcaatattattttaatgatgatggaattatgcaagttggatttgtcactataaatgataaagtcttctacttctctgactctggaattatagaatctggagtacaaaacatagatgacaattatttctatatagatgataatggtatagttcaaattggtgtatttgatacttcagatggatataaatattttgcacctgctaatactgtaaatgataatatttacggacaagcagttgaatatagtggtttagttagagttggggaagatgtatattattttggagaaacatatacaattgagactggatggatatatgatatggaaaatgaaagtgataaatattatttcaatccagaaactaaaaaagcatgcaaaggtattaatttaattgatgatataaaatattattttgatgagaagggcataatgagaacgggtcttatatcatttgaaaataataattattactttaatgagaatggtgaaatgcaatttggttatataaatatagaagataagatgttctattttggtgaagatggtgtcatgcagattggagtatttaatacaccagatggatttaaatactttgcacatcaaaatactttggatgagaattttgagggagaatcaataaactatactggttggttagatttagatgaaaagagatattattttacagatgaatatattgcagcaactggttcagttattattgatggtgaggagtattattttgatcctgatacagctcaattagtgattagtgaatag

SEQ ID NO: 10 - DNA, 2885 bp, Artificial Sequence
  C-TAB.G5.1
ccatggttacaggtgttttcaaaggtccgaacggctttgaatattttgcaccggcaaatacccacaataataatattgaaggccaggccatcgtgtatcagaataaatttctgaccctgaacggcaaaaaatactatttcgataacgatagcaaagcagttaccggttggcaaaccattgatggcaaaaaatattacttcaacctgaataccgcagaagcagcaaccggctggcagacgatcgacggtaaaaagtactattttaacctgaacacagccgaagccgctacaggctggcagacaatagatgggaagaagtattattttaataccaatacctttattgccagcaccggctataccagcattaatggcaaacacttctattttaacaccgatggtattatgcagatcggtgtgtttaagggccctaatggttttgagtacttcgctccggctaataccgatgcaaataacatcgaaggtcaggcaattctgtaccagaacaaatttttaacgctgaacggtaagaaatattactttggtagcgattcaaaagccgttaccggtctgcgtacgatcgacggcaagaaatattatttcaatacaaacaccgcagttgccgtgacaggttggcagacgataaatggtaagaagtactacttcaacaccaataccagcattgcaagtaccggttataccattatcagcggcaaacacttttacttcaatacagacggcattatgcagattggcgttttcaaaggtccggatggtttcgagtactttgcccctgcaaatacagatgcaaacaatattgagggacaggcaattcgctatcagaatcgttttctgtatctgcacgataacatctattacttcggcaataattcaaaagcagccaccggttgggttacaattgatggtaatcgttattactttgagccgaataccgcaatgggtgcaaatggttataaaaccatcgataacaaaaatttttatttccgcaacggtctgccgcagattggtgtttttaagggtagcaatggcttcgagtattttgcgccagccaacaccgatgccaacaacattgaaggccaagcgattcgttatcaaaaccgctttctgcatctgctgggcaaaatttattactttggcaacaatagcaaagcggtgacgggctggcaaaccattaacggtaaagtttattatttcatgccggataccgctatggcagcagccggtggtctgtttgaaattgatggcgtgatttatttttttggcgtggatggtgttaaagcaccgggtatttatggtcgtagcatgcataatctgattaccggttttgttaccgtgggcgacgataaatactactttaatccgattaatggtggtgcagcaagcattggtgaaaccattatcgatgacaaaaactattattttaaccagagcggtgttctgcagacaggtgtttttagcaccgaagatggcttcaaatattttgctcctgcgaatacactggatgaaaatctggaaggtgaagcaattgattttaccggcaaactgatcatcgacgagaacatctactattttgatgataattatcgcggtgccgtggaatggaaagaactggatggtgaaatgcactattttagtccggaaaccggtaaagcctttaaaggtctgaatcagatcggcgattacaagtattactttaattcagatggcgtgatgcagaaaggctttgtgagcattaacgacaacaaacactattttgacgacagcggtgtgatgaaagtgggttataccgaaatcgacgggaaacatttttattttgccgaaaacggcgaaatgcagattggagtatttaataccgaggacggctttaaatactttgcccatcataatgaagatctgggtaatgaagaaggcgaagaaattagctatagcggcattctgaattttaataacaagatctattatttcgatgatagcttcaccgcagttgttggttggaaagatctggaagatggcagcaaatattattttgatgaagataccgcagaggcctatattggtctgagcctgattaatgatggccagtattatttcaacgatgatggtatcatgcaggttggttttgtgaccatcaacgataaagtgttctatttcagcgatagcggcattattgaaagcggtgttcagaacatcgacgataactatttctacatcgatgataacggtattgttcagattggcgtgtttgatacctccgatggttataaatatttcgcaccagccaataccgtgaacgataatatttatggtcaggcagttgaatattcaggtctggttcgtgttggcgaagatgtttattattttggcgaaacctataccattgaaaccggctggatctatgatatggaaaacgagagcgacaagtactatttcaatccggaaacgaaaaaagcctgcaaaggcattaatctgatcgacgatattaagtactactttgacgaaaaaggcattatgcgtaccggtctgattagctttgagaacaacaactattacttcaatgagaacggtgagatgcagtttggctatatcaacatcgaggacaaaatgttttattttggtgaggacggtgtgatgcagataggggtttttaatacaccggatgggtttaagtattttgcacatcagaacaccctggatgaaaactttgaaggcgaaagcattaattataccggttggctggatctggatgagaaacgttattatttcaccgacgaatacattgcagcaaccggtagcgttattattgatggtgaggaatattacttcgatccggatacagcacagctggttattagcgaataactcgag

SEQ ID NO: 11 - protein, 21 aa, Artificial Sequence
  Peptide linker
GTSDKNNGSGSKEKNKDGKYS

SEQ ID NO: 12 - protein, 961 aa, Artificial Sequence
  Lip-C-TAB.G5.1
  MISC_FEATURE (1)..(1): Lipidation
CSSFVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYGRSMHNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 13 - protein, 449 aa, Artificial Sequence
  Lip-ToxA-His
  MISC_FEATURE (1)..(1): Lipidation
CSSFVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYGLEHHHHHH

SEQ ID NO: 14 - protein, 528 aa, Artificial Sequence
  Lip-ToxB-His
  MISC_FEATURE (1)..(1): Lipidation
CSSFNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISELEHHHHHH

SEQ ID NO: 15 - protein, 1276 aa, Artificial Sequence
  LIP-Protein\S_2019-nCoV/Italy-INMI1
  MISC_FEATURE (1)..(1): Lipidation
CSSMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 16 - protein, 523 aa, Artificial Sequence
  L7F_A1_23 protein sequence
MSWKVVIIFSLLITPQHGLKESYLEESCSTITEGYLSVLRTGWYTNVFTLEVGDVENLTCADGPSLIKTELDLTKSALRELRTVSADQLAREEQIEQPRQSGCGAGATAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTCAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSAGYIPEAPRDGQAYVRKDGEWVLLSTFLGGLVPRGSHHHHHHSAWSHPQFEK

SEQ ID NO: 17 - protein, 529 aa, Artificial Sequence
  sF_A1_K_L7 protein sequence
MSWKVVIIFSLLITPQHGLKESYLEESCSTITEGYLSVLRTGWYTNVFTLEVGDVENLTCADGPSLIKTELDLTKSALRELRTVSADQLAREEQIEQPRQSGCGAGATAGVAIAKTIRLESEVTAIKNALKKTNEAVSTLGNGVRVLAFAVRELKDFVSKNLTRALNKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTCAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAESAIGGYIPEAPRDGQAYVRKDGEWVLLSTFLGGLVPRGSHHHHHHSAWSHPQFEK

SEQ ID NO: 18 - protein, 523 aa, Artificial Sequence
  L7F_A1_31 protein sequence
MSWKVVIIFSLLITPQHGLKESYLEESCSTITEGYLSVLRTGWYTNVFMLEVGDVENLTCADGPSLLKTELDLTKSALRNLRTVSADQLAREEQIEQPRQSGCGAGATAGVAIAKTIRLESEVTAIKNALKKTNEAVSTLGNGVRVLATMVRELKDFVSKNLTRAINKNKCDIADLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTCAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSAGYIPEAPRDGQAYVRKDGEWVLLSTFLGGLVPRGSHHHHHHSAWSHPQFEK

SEQ ID NO: 19 - protein, 523 aa, Artificial Sequence
  L7F_A1_33 protein sequence
MSWKVVIIFSLLITPQHGLKESYLEESCSTITEGYLSVLRTGWYTNVFMLCVGDVENLTCADGPSLLKTELDLTKSALRELRTVSADQLAREEQIEQPRQSGCGAGATAGVAIAKTIRLESEVTAIKNALKKTNEAVSTLGNGVRVLATMVRELCDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSDVIYMVQLPIFGVIDTPCWIVKAAPSCSEKKGNYACLLREDQGWYCQNAGSTVYYPNEKDCETRGDHVFCDTCAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRCCSAGYIPEAPRDGQAYVRKDGEWVLLSTFLGGLVPRGSHHHHHHSAWSHPQFEK

SEQ ID NO: 20 - protein, 529 aa, Artificial Sequence
  L7F_A1_4.2 protein sequence
MSWKVVIIFSLLITPQHGLKESYLEESCSTITEGYLSVLRTGWYTNVFMLEVGDVENLTCADGPSLIKTELDLTKSALRELRTVSADQLAREEQIEQPRQSGCGAGATAGVAIAKTIRLESEVTAWKNALKKTNEVVSTLGNGVRVLVTMVRELKDFVSKNLTRALNKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTCAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAESAIGGYIPEAPRDGQAYVRKDGEWVLLSTFLGGLVPRGSHHHHHHSAWSHPQFEK

SEQ ID NO: 21 - protein, 540 aa, Artificial Sequence
  sF_A1_K-E294 protein sequence with substitutions  A113C, A339C, T160F, I177L and trimerization helper KLL
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVCTAAAVTAGVAIAKTIRLESEVTAIKNALKKTNEAVSTLGNGVRVLAFAVRELKDFVSKNLTRALNKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAESAIGGYIPEAPRDGQAYVRKDGEWVLLSTFLGGLVPRGSHHHHHHSAWSHPQFEK

SEQ ID NO: 22 - protein, 538 aa, Artificial Sequence
  sF_A1_MFur protein sequence with deletion of amino acids at positions 103 to 111, replacement of R102 by a furin site KKRKRR and the substitution G294E, stabilized in post- fusion conformation
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSKKRKRRVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGGGGSGYIPEAPRDGQAYVRKDGEWVLLSTFLGGIEGRHHHHHH

(19) United States
(12) Patent Application Publication
(10) Pub. No.: US 2023/0398210 A1
(43) Pub. Date: Dec. 14, 2023

(54) IMPROVEMENTS IN VACCINE FORMULATIONS FOR MEDICAL USE

(71) Applicant: Valneva Austria GmbH, Vienna (AT)

(72) Inventors: Robert Schlegl, Siegenfeld (AT); Jürgen Heindl-Wruss, Vienna (AT)

(73) Assignee: Valneva Austria GmbH, Vienna (AT)

(21) Appl. No.: 17/917,010

(22) PCT Filed: Apr. 9, 2021
(86) PCT No.: PCT/EP2021/059333

§ 371 Date: Oct. 5, 2022

(30) Foreign Application Priority Data
Apr. 9, 2020 (EP) 20169147.4
Apr. 9, 2020 (EP) 20169148.2
Apr. 15, 2020 (EP) 20169642.4
Jul. 22, 2020 (EP) 20187283.5
Oct. 20, 2020 (EP) 20202816.3

(51) Int. Cl. A61K 39/39; A61K 47/20; A61K 39/02; A61K 47/02

(52) U.S. Cl. CPC A61K 39/39; A61K 47/20; A61K 39/0225; A61K 47/02; A61K 2039/55505

(57) ABSTRACT

The present invention relates to an aluminum composition for use as an adjuvant or for use in a method of vaccination, the use of an alum-adjuvanted vaccine composition comprising less than 1.25 ppb copper for increasing bioavailability of an antigen in the vaccine, wherein the antigen is a protein, particularly an OspA protein or a Clostridium difficile toxin A and toxin B fusion protein or a SARS-CoV-2 protein or an hMPV protein, in an aluminum-containing vaccine composition and the use of a radical quenching compound such as L-methionine for increasing bioavailability of a protein antigen in a copper- and aluminum-containing vaccine composition.

### RELATED APPLICATION

This application is a national stage filing under 35 U.S.C. 371 of International Patent Application Serial No. PCT/EP2021/059333, filed Apr. 9, 2021, the content of which is incorporated herein by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Oct. 4, 2022, is named I042270144US00-SEQ-JRV and is 97,403 bytes in size.

### FIELD OF THE INVENTION

The present invention relates to an aluminum composition for use as an adjuvant or for use in a method of vaccination, the use of an alum-adjuvanted vaccine composition comprising less than 1.25 ppb copper for increasing bioavailability of an antigen in the vaccine, wherein the antigen is a protein, particularly an OspA protein or a Clostridium difficile toxin A and toxin B fusion protein or a SARS-CoV-2 protein or an hMPV protein, in an aluminum-containing vaccine composition and the use of a radical quenching compound such as L-methionine for increasing bioavailability of a protein antigen in a copper- and aluminum-containing vaccine composition.

### BACKGROUND OF THE INVENTION

Aluminium compounds (herein also referred to as “aluminum”), including aluminium phosphate (AlPO₄), aluminium hydroxide (Al(OH)₃), and other aluminium preparations are currently the most commonly used adjuvants for human and veterinary vaccines. The adjuvants are often referred to as “alum” in the literature.

Aluminium adjuvants have been used in practical vaccination for more than half a century. They induce early, high-titer, long-lasting protective immunity. Billions of doses of aluminium-adjuvanted vaccines have been administered over the years. Their safety and efficacy have made them the most popular adjuvants in vaccines to date. In general, aluminium adjuvants are regarded as safe when used in accordance with current vaccination schedules.

Historically, aluminium adjuvants have been used in e.g. tetanus, diphtheria, pertussis and poliomyelitis vaccines as part of standard child vaccination programs. Aluminium adjuvants have also been introduced into hepatitis A and hepatitis B virus vaccines and Japanese encephalitis virus (also referred to herein as “JEV”) vaccines. Other aluminium-adsorbed vaccines against, for example, anthrax, are available for special risk groups. In veterinary medicine, aluminium adjuvants have been used in a large number of vaccine formulations against viral and bacterial diseases, and in attempts to make anti-parasite vaccines.

Adjuvants typically serve to bring the antigen, the substance that stimulates the specific protective immune response, into contact with the immune system and to influence the type of immunity produced, as well as the quality of the immune response (magnitude and/or duration). Adjuvants can also decrease the toxicity of certain antigens and provide solubility to some vaccines components. Studies have shown that many aluminium-containing vaccines stimulate stronger and more prolonged antibody responses than comparable vaccines without an adjuvant. The benefit of adjuvants is particularly pronounced during the initial immunization series as compared with booster doses.

There are three general types of aluminium-containing adjuvants: Aluminium hydroxide, Aluminium phosphate and Potassium aluminium sulphate (collectively often referred to as “alum”). The effectiveness of each salt as an adjuvant depends on the characteristics of the specific vaccine antigen and how it is manufactured. To work as an adjuvant, the antigen is typically adsorbed to the aluminium particles; that is, it is complexed with the aluminium salt to keep the antigen at the site of injection.

In EP2869839, it has been shown that stability of a biological in a composition that also comprises an aluminium salt is not always the same. For instance, it was shown that the stability of a protein component (e.g. as such or within a complex such as e.g. a virus or other pathogen) in the context of an aqueous composition that also comprises aluminium salt is dependent on the content of heavy metals and in particular copper. In comparison to the disclosure of EP2869839, the current invention discloses reduced bioavailability of protein antigens in alum preparations with heavy metal contaminants. Specifically, aqueous compositions, such as vaccines of this invention, which comprise a protein antigen and aluminium salts (alum) show reduced desorption of the protein antigen from alum (i.e. the bioavailability is reduced) when the alum comprises high amounts of heavy metals, e.g. a copper amount of higher than 1.25 ppb in said aqueous composition. This surprising lack of bioavailability effect for protein vaccines of the invention has not been observed for vaccine compositions of EP2869839. Methods and uses to increase the bioavailability of alum adjuvanted antigens are herein described.

### SUMMARY OF THE INVENTION

During the course of the current invention, it was observed that formulation with alum with a high heavy metal content and/or a high copper content resulted in reduced protein recovery following desorption from alum. The reduced recovery may be due to protein degradation and/or protein modifications resulting in changes to protein-alum binding strength. Both scenarios can result in decreased bioavailability of antigen in vaccinated subjects. Strong protein-alum binding has been shown in some cases to result in reduced immunogenicity (Egan et al., Vaccine 27 (2009) 3175-3180; Hansen et al., Vaccine 27 (2009) 888-892; Hansen et al., Vaccine 25 (2007) 6618-6624; Noe at al., Vaccine 28 (2010) 3588-3594). Differences in alum quality in a vaccine may significantly affect the consistency of efficacy from lot to lot. Two solutions are presented herein: 1) to formulate using alum preparations with a low heavy metal content, particularly a low copper content, and/or 2) to provide a quencher such as e.g. L-methionine in the formulation.

### BRIEF DESCRIPTION OF THE DRAWINGS

FIG. 1. Desorption recovery of Lip-S5D1-S6D1 antigen from formulations as described in Table 2 with two different alum lots and A) no additives, B) L-methionine, C) sulfite, and D) L-methionine and sulfite.

FIG. 2. RP-HPLC overlay of desorbed protein after 17 days incubation at +37° of Lip-S5D1-S6D1 formulations as prepared in Table 2 with two different alum lots and A) no additives, B) L-methionine, C) sulfite, and D) L-methionine and sulfite.

### DETAILED DESCRIPTION OF THE INVENTION

Therefore, in a first aspect, the present invention provides an aluminum composition for use as an adjuvant, wherein the composition (i) comprises less than a sufficiently low amount of copper such as e.g. 1.25 ppb copper or a sufficient amount of a radical quenching compound such as e.g. at least as the amount of L-methionine and (ii) increases the bioavailability of an antigen in the vaccine, wherein the antigen is a protein, such as e.g. an OspA protein, a Clostridium difficile toxin a SARS_CoV-2 spike protein or an hMPV F protein. Particularly, the present invention provides an aluminum composition for use as an adjuvant, wherein the composition comprises less than a sufficiently low amount of copper such as e.g. 1.25 ppb copper and increases the bioavailability of an antigen in the vaccine, wherein the antigen is a protein, such as e.g. an OspA protein, a Clostridium difficile toxin a SARS_CoV-2 spike protein or an hMPV F protein. Alternatively, the present invention provides an aluminum composition for use as an adjuvant, wherein the composition comprises a sufficient amount of L-methionine and increases the bioavailability of an antigen in the vaccine, wherein the antigen is a protein, such as e.g. an OspA protein, a Clostridium difficile toxin, a SARS-CoV-2 spike protein or an hMPV F protein.

The first protein-based vaccines relied on natural sources of antigens. To develop a vaccine, plasma was harvested from long-term chronic hepatitis B carriers, Hepatitis B surface antigen (HBsAg) purified, and the final preparation subjected to one to three inactivation techniques (depending on the manufacturer) to kill HBV and any other adventitious human agents possibly present in the starting plasma. This vaccine was well tolerated and highly efficacious, but is no longer used as the vaccine can now be made by recombinant technology.

Recombinant or purified protein vaccines consist of protein antigens that have either been produced in a heterologous expression system (e.g., bacteria or yeast) or purified from large amounts of the pathogenic organism. The vaccinated person produces antibodies to the protein antigen, thus protecting him/her from disease.

Protein vaccines are based on the concept that humoral immune responses mounted to an infection are often targeted toward specific localized regions on the surface of protein antigens known as epitopes. The recombinant proteins for vaccination are produced by expressing these immunogenic proteins using heterologous expression systems. The immunogenic protein antigens can also be purified from the infectious organism. Once purified, protein antigens, recombinant and endogenous, are administered with an adjuvant to boost the immune response. Administering only the most immunogenic protein or proteins from an infectious organisms as a vaccine produces a more targeted immune response. This strategy also eliminates the risk of active infection that can occur with live attenuated vaccines or even inactivated vaccines where inactivation is incomplete.

Exemplary protein vaccines include cholera toxin B as a component of Dukoral, chemically inactivated diphtheria toxin, SARS-CoV-2 spike protein, Clostridium difficile toxins, hepatitis B surface antigen, inactivated tetanus toxin, hMPV protein and Borrelia OspA proteins and variants thereof.

Lyme borreliosis (LB) is an emerging infectious disease transmitted by ticks in the northern hemisphere. Different vaccine candidates based on the Outer surface protein A (OspA) of Borrelia species have been developed, which are referred to as OspA proteins in accordance with the present invention. As monovalent OspA-based vaccines preclude efficient protection with a vaccine based on OspA from only a single serotype due to heterogeneity in OspA sequences across different serotypes in Europe and elsewhere, chimeric OspA molecules have been developed. Chimeric OspA molecules comprising the proximal portion from one OspA serotype, together with the distal portion form another OspA serotype, while retaining antigenic properties of both of the parent polypeptides, may be used in the vaccination of Lyme disease or borreliosis. Such OspA proteins are described in WO2011/143617 and WO2011/143623. Moreover, it was found that the introduction of at least one disulfide bond in mutant fragments of OspA increases the protective capacity of the polypeptide comprising the mutant OspA fragment relative to a polypeptide comprising the wild-type OspA fragment (WO2014/006226). Moreover, hybrid C-terminal OspA fragments have been developed, wherein the hybrid fragment consists of a C-terminal domain of an OspA protein of Borrelia that is comprised of a fragment derived from an OspA protein of a Borrelia strain different than B. garinii, strain PBr, and a second fragment of OspA from B. garinii, strain PBr, and differs from the corresponding wild-type sequence at least by the introduction of at least one disulfide bond (WO2015/104396). Further, immunogenic polypeptides comprising a stabilized C-terminal OspA domain comprising two or more specific OspA epitopes each from distinct Borrelia strains causing Lyme borreliosis and being able to induce a protective immune response to all of said distinct Borrelia strains have been provided (WO2018/189372). Particularly, any of the OspAs provided in the above applications may be used as a vaccine antigen in the context of the present invention. Particularly preferred is the previously-described multimeric Borrelia OspA vaccine, in which the C-terminal parts, or hybrid C-terminal parts, of two monomers of the six OspA serotypes (ST1 to ST6) stabilized with disulfide bonds were linked together in each of the three fusion proteins (further details are also given below). Preferably, the OspA protein is lipidated.

Clostridium difficile is the leading cause of nosocomial antibiotic associated diarrhea and has become a major health problem in hospitals, nursing home and other care facilities. C. difficile associated disease (CDAD) is induced by the disruption of the normal colonic flora, usually the result of the administration of antibiotics. Following exposure to C. difficile spores in the environment, the organism may colonize the intestinal mucosa where the production of disease causing toxins can result in CDAD. Disease may range from mild uncomplicated diarrhea to severe pseudomembranous colitis and toxic megacolon. CDAD is the result of the actions of two exotoxins produced by C. difficile, toxin A and toxin B (also referred to as CTA and CTB, respectively). Both toxins are high molecular weight (˜300 kDa) secreted proteins that possess multiple functional domains (Voth D E and Ballard J D, Clinical Microbiology Reviews 18:247-263 (2005)). The N-terminal domain of both toxins contains ADP-glucosyltransferase activity that modifies Rho-like GTPases. This modification causes a loss of actin polymerization and cytoskeletal changes resulting in the disruption of the colonic epithelial tight junctions. This leads to excessive fluid exudation into the colon and a resulting diarrhea. The central domain contains a hydrophobic domain and is predicted to be involved in membrane transport. The C-terminal domain of both toxins contains multiple homologous regions called repeating units (RUs) that are involved in toxin binding to target cells (Ho et al, (2005) PNAS 102(51):18373-18378). The repeating units are classified as either short (21-30 amino acids) or long (˜50 amino acids). Repeating units combine to form clusters, each usually containing one long and 3-5 short repeating units. The full-length toxin A possesses 39 repeating units (ARUs) organized into 8 clusters (Dove et al. Infect. Immun. 58:480-488 (1990)), while the full-length toxin B contains 24 repeating units (BRUs) organized into 5 clusters (Barroso et al., Nucleic Acids Res. 18:4004 (1990); Eichel-Streiber et al., Gene 96:107-113 (1992)). Further details on suggested Clostridium difficile toxin protein based vaccines may be found in WO2012028741A1 and EP2753352B2. In one embodiment, the Clostridium difficile toxin protein is lipidated.

SARS-CoV-2 was detected for the first time in China around November 2019. Since then, the virus has caused a global pandemic. The natural reservoir are bats and the virus belongs to the Coronaviridae family, genus Betacoronavirus (betaCoV). The virus has a ssRNA genome composed of 29,903 bp (Wuhan-Hu-1: Genbank Reference sequence: NC_045512.2), which encode a 9,860 amino acid polyprotein, comprising 25 non-structural proteins and 4 structural proteins: spike (S), envelope (E), membrane (M) and nucleocapsid (N) proteins. The spike protein is a particularly preferred target for a vaccine. SARS-CoV-2 presents a substantial public health threat. The Imperial College COVID-19 (disease caused by SARS-CoV-2) Response Team published in Mar. 16, 2020, a report evaluating all possible methods available to stop or delay the spread of the virus, which could ultimately lead to the break-down of the healthcare system and hundreds of thousands of deaths in the UK alone. The report stated that only population-wide social distancing has a chance to reduce effects to manageable levels and these measures need to be followed until a vaccine is available. This recommendation would mean for most of the population quarantine for at least 18 months. The report concluded that a mass-producible vaccine is the only option to stop this pandemic, other than a willingness to sacrifice the elderly population. In view of the dramatic situation, there is an absolute urgent need for an effective vaccine against SARS-CoV-2 as fast as possible. Furthermore, various escape mutants have emerged (e.g. UK_B.1.1.7; South African_B.1.351; Californian_B.1.427/B.1.429 and Brazilian_P.1 variants) which further worsen the situation and thus addressing this unfortunate development needs to be a priority as well.

Human metapneumovirus (hMPV) is a leading cause of acute respiratory tract infections in young children (0-4 years), immunocompromised patients and in elderly that can be fatal for these categories of patients (Schildgen et al. 2011. Clinical Microbiology Reviews 24(4): 734-54). hMPV is an enveloped, single-stranded RNA virus of the genus Pneumovirus of the family Paramyxoviridae. The hMPV genome consists of eight genes encoding nine proteins, including three surface glycoproteins F, G and SH. Protection against hMPV is afforded mainly by neutralizing antibodies directed against the fusion (F) glycoprotein, which is highly conserved between different genotypes and shares similarities to other paramyxoviruses (see van den Hoogen et al. 2004. Emerging Infectious Diseases 10(4): 658-66; van den Hoogen et al. 2002. Virology 295(1): 119-32). Among several vaccination strategies investigated, a subunit vaccine containing a viral protein, especially the hMPV F protein, is the most promising (Melero & Mas. 2015. Virus Res. 209: 128-35). Paramyxoviral F protein is a type I integral membrane protein that spans the membrane once and contains at its N-terminus a signal peptide, which targets the ectodomain to the extracellular membrane. At the C-terminus, a hydrophobic stop-transfer domain (TM domain) anchors the protein in the membrane, leaving a short cytoplasmic tail. Further details on hMPV and suitable protein vaccines are derivable from WO 2020/234300 A1.

In the lipidated proteins of the invention, the lipid moieties, along with the glycerol group, is also referred to as “Lip”. According to the invention, Lip comprises one to three lipids such as C₁₄₋₂₀ alkyl and/or C₁₄₋₂₀ alkenyl attached to a glycerol and an amino group of the N-terminal cysteine of the polypeptide of the invention, or preferably wherein Lip is a moiety of formula (I) below,

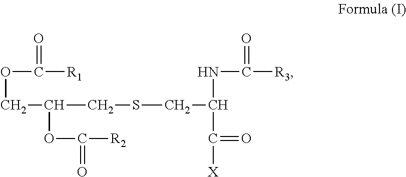

in which one of R₁, R₂ or R₃ is C₁₄-C₂₀ alkyl or alkenyl, and each of the others, independently is C₁₄-C₂₀ alkyl or C₁₄-C₂₀ alkenyl, and X is an amino acid sequence attached to the cysteine residue shown in Formula (I). More preferably, Lip plus the N-terminal cysteine of the polypeptide is N-palmitoyl-S-(2RS)-2,3-bis-(palmitoyloxy) propyl cysteine (referred to herein as “Pam₃Cys”) and is connected via the carbonyl C of the cysteine to said amino acid sequence of the invention. In Formula (I) above R₁, R₂ and R₃ would be palmitoyl moieties and X is an amino acid sequence attached to the cysteine residue.

The vaccine protein is encompassed in a composition. The composition is pharmaceutically acceptable, which allows for administration to a human. It may optionally contain any pharmaceutically acceptable carrier or excipient, such as buffer substances, stabilizers or further active ingredients, especially ingredients known in connection with pharmaceutical compositions and/or vaccine production. The composition may comprise sodium phosphate, sodium chloride, L-methionine, sucrose and Polysorbate-20 (Tween 20) at a pH of 6.7+/−0.2. Preferably, the pharmaceutical composition also comprises aluminium hydroxide, preferably at a concentration of 0.15%. Additionally, the composition may comprise between 5 mM and 50 mM sodium phosphate, between 100 and 200 mM sodium chloride, between 5 mM and 25 mM L-methionine, between 2.5% and 10% Sucrose, between 0.01% and 0.1% Tween 20 and between 0.1% and 0.2% (w/v) aluminium hydroxide. More preferably, the formulation comprises 10 mM sodium phosphate, 150 mM sodium chloride, 10 mM L-methionine, 5% Sucrose, 0.05% Tween 20 and 0.15% (w/v) aluminium hydroxide at pH 6.7±0.2. In a preferred embodiment, the excipient is L-methionine.

According to the invention, the OspA protein is used for vaccination, particularly against an infection caused by Borrelia species, more preferably pathogenic Borrelia species as disclosed herein more preferably comprising B. burgdorferi s.s., B. afzelii, B. bavariensis and B. garinii, and/or other pathogens against which the antigens have been included in the vaccine. Preferably, the Borrelia species is selected from B. burgdorferi s.s., B. garinii, B. afzelii, B. andersoni, B. bavariensis, B. bissettii, B. valaisiana, B. lusitaniae, B. spielmanii, B. japonica, B. tanukii, B. turdi or B. sinica infection, preferably a B. burgdorferi s.s., B. afzelii, B. bavariensis, B. mayonii and B. garinii.

According to the invention, the Clostridium difficile toxin protein is used for vaccination, particularly against an infection caused by Clostridium difficile.

According to the invention, the spike protein of SARS-CoV-2 is used for vaccination, particularly against an infection caused by SARS-CoV-2 (i.e. COVID-19).

According to the invention, the hMPV F protein is used for vaccination, particularly against an infection caused by hMPV.

The composition according to the present invention comprises the antigenic protein and the adjuvant. It may be administered to the human as an injectable composition, for example as a sterile aqueous dispersion, preferably isotonic. The composition may be administered via a systemic or mucosal route. These administrations may include injection via the intramuscular, intraperitoneal, intradermal or subcutaneous routes; or via mucosal administration to the oral/alimentary, respiratory or genitourinary tracts. Although the vaccine of the invention may be administered as a single dose, components thereof may also be co-administered together at the same time.

In the process of vaccination, a protein, such as an OspA protein or a Clostridium difficile toxin protein or a SARS-CoV-2 protein, particularly the spike protein or fragment thereof, or an hMPV F protein, is administered along with an aluminum composition, which is used as an adjuvant. An adjuvant is a substance that acts to accelerate, prolong, or enhance antigen-specific immune responses when used in combination with specific vaccine antigens. Aluminum compounds, particularly aluminum salts, are well-known adjuvants. Aluminum phosphate (AlPO₄), aluminum hydroxide (Al(OH)₃), and other aluminum precipitated vaccines are currently the most commonly used adjuvants with human and veterinary vaccines. The adjuvants are often referred to as “alum” in the literature.

Aluminum adjuvants have been used in practical vaccination for more than half a century. They induce early, high-titer, long-lasting protective immunity. Billions of doses of aluminium-adjuvanted vaccines have been administered over the years. Their safety and efficacy have made them the most popular adjuvants in vaccines to date. In general, aluminum adjuvants are regarded as safe when used in accordance with current vaccination schedules. In human vaccinations, historically, aluminum adjuvants have been used, e.g., in tetanus, diphtheria, pertussis and poliomyelitis vaccines as part of standard child vaccination programs.

Adjuvants typically serve to bring the antigen, the substance that stimulates the specific protective immune response, into contact with the immune system and influence the type of immunity produced, as well as the quality of the immune response (magnitude or duration). Adjuvants can also decrease the toxicity of certain antigens; and provide solubility to some vaccines components. Studies have shown that many aluminium-containing vaccines cause higher and more prolonged antibody responses than comparable vaccines without the adjuvant. The benefit of adjuvants has usually been observed during the initial immunization series rather than with booster doses. There are three general types of aluminium-containing adjuvants: aluminium hydroxide, aluminium phosphate and potassium aluminium sulphate (collectively often referred to as “alum”). To work as an adjuvant, the antigen is typically adsorbed to the aluminium particles; that is, it is complexed with the aluminium salt to keep the antigen at the site of injection.

The aluminum adjuvants typically comprise impurities, particularly heavy metals, such as copper, nickel and iron. As explained above and shown in the Examples, the presence of these, particularly of copper, lowers the bioavailability of the OspA protein in the vaccine. Without being bound to the theory, it is assumed that the (OspA) protein binds to the aluminum and the heavy metal, particularly the copper, preventing antigen release, and thus decreasing the bioavailability of the protein (OspA) in the vaccine. Therefore, the adjuvant preferably comprises less than 1.25 ppb copper.

The unit ppb (parts per billion) is often used in the field of mass spectrometry to quantify impurities. In case of aqueous solutions, 1 ppb means that 1 ng of substance (impurity) is present in 1 g solution, which means that 1 ppb equals 1 μg/l (assuming that 1 liter of solution has a weight of 1 kg).

In a preferred embodiment, the composition comprises less than 1.00 ppb, less than 0.75 ppb or less than less than 0.50 ppb copper based on the weight of the aqueous composition.

Preferably, the adjuvant further comprises no more than 350 ppb of a heavy metal based on the weight of the aqueous composition. Typical heavy metal impurities include Cu, Ni, W, Co, Os, Ru, Cd, Ag, Fe, V, especially Fe, Ni and Cu. Preferably the amount of heavy metal impurities in the aqueous composition is less than 250 ppb, preferably less than 225, more preferably less than 200, more preferably less than 150, more preferably less than 100, more preferably less than 50, more preferably less than 25 ppb based on the weight of the aqueous composition. This amount is typically for the total of determined heavy metals, or for the heavy metals Fe, Cr and Ni, or a combination thereof which constitute the major heavy metals by weight in the aqueous composition of the invention. For specific heavy metals different maximums amounts may be preferred. For instance, it is preferred that the amount of Fe in the aqueous composition of the invention is less than 350 ppb based on the weight of the aqueous composition. In a preferred embodiment the amount of Fe is less than 250 ppb, preferably less than 210 ppb Fe based on the weight of the aqueous composition.

In the event that the alum-adjuvanted composition of the invention comprises more than 1.25 ppb copper, a sufficient amount of a radical quenching compound may be added to the composition. It is known that radical quenching compounds, such as e.g. L-methionine, are capable of binding to copper. It is assumed that L-methionine first interacts with the copper via its sulfur, oxygen and nitrogen atoms. After binding to L-methionine, the influence of copper to the binding of the protein (OspA) to aluminum is reduced, thus increasing the bioavailability of the vaccine. The amount of L-methionine or other quenching compound required will evidently depend on the amount of copper in the composition. The person skilled in the art will be capable of selecting a suitable amount of e.g. L-methionine.

A radical quenching compound is a chemical substance added to a mixture in order to remove or de-activate impurities and unwanted reaction products, in the present invention particularly heavy metals, especially copper, to avoid any unfavorable reactions of activities. Exemplary radical quenching compounds include thiol, DMT, AMPA, imidazole, cysteine, thio-urea and L-methionine. Preferably, the radical quenching compound is L-methionine.

In a second aspect, the present invention provides an aluminum composition for use in a method of vaccination, wherein the composition (i) comprises less than 1.25 ppb copper or a sufficient amount of a radical quenching compound and (ii) increases the bioavailability of an antigen in the vaccine, wherein the antigen is a protein, such as e.g. an OspA protein, a Clostridium toxin fusion protein, a spike protein of SARS-CoV-2 and/or an hMPV F protein.

The above definitions and comments made with respect to the first aspect of the invention apply also to the second aspect of the invention.

In the following preferred embodiments of the first and second aspect of the present invention are described.

Preferably, the protein in the vaccine is an OspA protein. More preferably, the OspA protein in the above composition is an OspA heterodimer. As detailed above, the OspA heterodimers are mutant OspA fragment heterodimers comprising disulfide-stabilized C-terminal OspA fragments and/or a hybrid C-terminal OspA fragment, wherein the hybrid fragment consists of a C-terminal domain of an OspA protein of Borrelia that is comprised of a fragment derived from an OspA protein of a Borrelia strain different than B. garinii, strain PBr, and a second fragment of OspA from B. garinii, strain PBr, and differs from the corresponding wild-type sequence at least by the introduction of at least one disulfide bond. The disulfide bonds of the C-terminal OspA fragments and the hybrid C-terminal fragment are disulfide bonds Type 1, e.g. cysteine residues are inserted at position 182+/−3 and 269+/−3 (for further details see WO2014/006226 and WO 2015/104396 A1). S3hyb indicates a fusion of amino acids 125-176 of B. valaisiana and amino acids 177-274 of B. garinii, strain PBr. Lip means lipidation and indicates the N-terminal addition of glycerol and fatty acid residues. The “LN1” peptide linker is a fusion of two separate loop regions of the N-terminal half of OspA from B. burgdorferi s.s., strain B31 (aa 65-74 and aa 42-53, with an amino acid exchange at position 53 of D53S) which has the following sequence: GTSDKNNGSGSKEKNKDGKYS (SEQ ID NO: 11). Preferably, the OspA proteins are lipidated.

Most preferably, the OspA vaccine comprises the heterodimer protein of SEQ ID NO: 1 (Lip-S1D1-S2D1), the heterodimer protein of SEQ ID NO: 2 (Lip-S4D1-S3hybD1) and the heterodimer protein of SEQ ID NO: 3 (Lip-S5D1-S6D1).

Lip-S1D1-S2D1 is a heterodimer fusion protein of an OspA serotype 1 fragment and OspA serotype 2 fragment, each with a disulfide bond type 1, the heterodimer comprising an N-terminal CSS for addition of lipids, a LN1 linker sequence and an N-terminal lipidation. Amino acids 164-174 of the OspA serotype 1 fragment are replaced by non-hLFA-1-like sequence NFTLEGKVAND. The sequence is shown as follows:

Lip-S1D1-S2D1-aa
SEQ ID NO: 1
LipCSSFNEKGEVSEKIITRADGTRLEYTGIKSDGSGKAKEVLKNFTLE
GKVANDKTTLVVKCGTVTLSKNISKSGEVSVELNDTDSSAATKKTAAWN
SGTSTLTITVNSKKTKDLVFTKENTITVQQYDSNGTKLEGSAVEITKLD
EICNALKGTSDKNNGSGSKEKNKDGKYSFNEKGELSAKTMTRENGTKLE
YTEMKSDGTGKAKEVLKNFTLEGKVANDKVTLEVKCGTVTLSKEIAKSG
EVTVALNDTNTTQATKKTGAWDSKISTLTISVNSKKTTQLVFTKQDTIT
VQKYDSAGTNLEGTAVEIKTLDELCNALK

Lip-S4D1-S3hybD1 is a heterodimer fusion protein of an OspA serotype 4 fragment and a hybrid OspA serotype 3 fragment, which hybrid comprises amino acids 125-176 of B. valaisiana, strain VS116 and amino acids 177-274 of B. garinii, strain PBr, serotype 3, each with a disulfide bond type 1, the heterodimer comprising an N-terminal CSS for addition of lipids, an LN1 linker sequence and N-terminal lipidation. The sequence is shown as follows:

Lip-S4D1-S3hybD1-aa
SEQ ID NO: 2
LipCSSFNAKGELSEKTILRANGTRLEYTEIKSDGTGKAKEVLKDFALE
GTLAADKTTLKVTCGTVVLSKHIPNSGEITVELNDSNSTQATKKTGKWD
SNTSTLTISVNSKKTKNIVFTKEDTITVQKYDSAGTNLEGNAVEIKTLD
ELCNALKGTSDKNNGSGSKEKNKDGKYSFNEKGEVSEKILTRSNGTTLE
YSQMTDAENATKAVETLKNGIKLPGNLVGGKTKLTVTCGTVTLSKNISK
SGEITVALNDTETTPADKKTGEWKSDTSTLTISKNSQKTKQLVFTKENT
ITVQNYNRAGNALEGSPAEIKDLAELCAALK

Lip-S5D1-S6D1 is a heterodimer fusion protein of an OspA serotype 5 fragment and an OspA serotype 6 fragment, each with a disulfide bond type 1, the heterodimer comprising an N-terminal CSS for addition of lipids, LN1 linker sequence and an N-terminal lipidation. The sequence is shown as follows:

Lip-S5D1-S6D1-aa
SEQ ID NO: 3
LipCSSFNEKGEISEKTIVRANGTRLEYTDIKSDKTGKAKEVLKDFTLE
GTLAADGKTTLKVTCGTVTLSKNISKSGEITVALDDTDSSGNKKSGTWD
SGTSTLTISKNRTKTKQLVFTKEDTITVQNYDSAGTNLEGKAVEITTLK
ELCNALKGTSDKNNGSGSKEKNKDGKYSFNGKGETSEKTIVRANGTRLE
YTDIKSDGSGKAKEVLKDFTLEGTLAADGKTTLKVTCGTVVLSKNILKS
GEITAALDDSDTTRATKKTGKWDSKTSTLTISVNSQKTKNLVFTKEDTI
TVQRYDSAGTNLEGKAVEITTLKELCNALK

The nucleic acid sequences encoding the above heterodimers are as follows:

Lip-S1D1-S2D1-nt
SEQ ID NO: 4
ATGAAAGCTACTAAACTGGTACTGGGCGCGGTAATCCTGGGTTCTACTCTGCTGGCAGGTT
GCTCAAGCTTCAACGAAAAGGGCGAAGTCAGCGAAAAAATCATTACCCGCGCAGACGGCAC
CCGCCTGGAATACACCGGCATCAAATCGGACGGCAGCGGCAAAGCGAAAGAAGTTCTGAAA
AACTTTACCCTGGAAGGCAAAGTCGCAAATGATAAAACCACCCTGGTGGTGAAATGCGGCA
CCGTTACGCTGAGCAAAAACATTAGTAAATCCGGTGAAGTCTCTGTGGAACTGAATGATAC
CGACAGCTCTGCGGCCACCAAGAAAACCGCAGCTTGGAACTCAGGCACCTCGACGCTGACC
ATTACGGTTAATAGCAAGAAAACCAAAGATCTGGTCTTCACGAAAGAAAACACCATCACGG
TGCAGCAATATGACAGCAATGGTACCAAACTGGAAGGCTCCGCTGTGGAAATCACGAAACT
GGATGAAATCTGTAATGCTCTGAAAGGTACTAGTGACAAAAACAATGGCTCTGGTAGCAAA
GAGAAAAACAAAGATGGCAAGTACTCATTCAACGAAAAAGGCGAACTGTCGGCGAAAACGA
TGACGCGTGAAAACGGCACCAAACTGGAATATACGGAAATGAAAAGCGATGGCACCGGTAA
AGCGAAAGAAGTTCTGAAAAACTTTACCCTGGAAGGCAAAGTCGCCAATGACAAAGTCACC
CTGGAAGTGAAATGCGGCACCGTTACGCTGTCAAAAGAAATTGCAAAATCGGGTGAAGTGA
CCGTTGCTCTGAACGATACGAATACCACGCAAGCGACCAAGAAAACCGGCGCCTGGGACAG
CAAAACCTCTACGCTGACCATTAGTGTTAATAGCAAGAAAACCACGCAGCTGGTCTTCACC
AAACAAGATACGATCACCGTGCAGAAATACGACAGTGCGGGTACCAACCTGGAAGGCACGG
CTGTTGAAATCAAAACCCTGGACGAACTGTGTAACGCCCTGAAA
Lip-S4D1-S3hybD1-nt
SEQ ID NO: 5
ATGAAAGCTACTAAACTGGTACTGGGCGCGGTAATCCTGGGTTCTACTCTGCTGGCAGGTT
GCTCAAGCTTCAATGCTAAGGGCGAACTGAGCGAAAAAACGATCCTGCGTGCGAATGGCAC
CCGTCTGGAATACACCGAAATCAAATCCGATGGTACGGGCAAAGCAAAGGAAGTCCTGAAA
GATTTTGCTCTGGAAGGTACCCTGGCGGCCGACAAAACCACGCTGAAGGTGACGTGCGGCA
CCGTGGTTCTGAGCAAACATATTCCGAACTCTGGTGAAATCACCGTTGAACTGAACGATAG
CAATTCTACGCAGGCAACCAAAAAGACGGGCAAATGGGACAGTAATACCTCCACGCTGACC
ATTTCAGTCAACTCGAAAAAGACCAAAAATATTGTGTTCACGAAGGAAGATACGATCACCG
TTCAAAAATATGACTCCGCGGGCACCAACCTGGAAGGCAATGCCGTCGAAATCAAAACCCT
GGATGAACTGTGTAACGCCCTGAAGGGTACTAGTGACAAAAACAATGGCTCTGGTAGCAAA
GAGAAAAACAAAGATGGCAAGTACTCATTCAACGAAAAAGGCGAAGTGAGCGAAAAAATTC
TGACCCGTAGCAATGGCACCACCCTGGAATATAGCCAGATGACCGATGCAGAAAATGCAAC
CAAAGCAGTTGAAACCCTGAAAAACGGTATTAAACTGCCTGGTAATCTGGTTGGTGGTAAA
ACCAAACTGACCGTTACCTGTGGCACCGTTACCCTGAGCAAAAACATTAGCAAAAGCGGTG
AAATTACCGTGGCACTGAATGATACCGAAACCACACCGGCAGACAAAAAAACCGGTGAATG
GAAAAGCGATACCAGCACCCTGACCATTAGTAAAAATAGCCAGAAAACAAAACAGCTGGTG
TTTACCAAAGAAAACACCATTACCGTGCAGAATTATAACCGTGCAGGTAATGCACTGGAAG
GTAGTCCGGCAGAAATTAAAGATCTGGCAGAACTGTGTGCAGCCCTGAAATAA
Lip-S5D1-S6D1-nt
SEQ ID NO: 6
ATGAAAGCTACTAAACTGGTACTGGGCGCGGTAATCCTGGGTTCTACTCTGCTGGCAGGTT
GCTCAAGCTTCAACGAAAAGGGCGAAATCTCAGAAAAAACCATCGTCCGCGCTAACGGCAC
CCGCCTGGAATACACCGACATCAAATCAGACAAGACCGGTAAAGCGAAGGAAGTTCTGAAA
GATTTTACGCTGGAAGGTACCCTGGCAGCAGACGGTAAAACCACGCTGAAGGTGACCTGCG
GTACCGTTACGCTGTCCAAAAACATTAGTAAGTCCGGCGAAATCACGGTCGCCCTGGATGA
CACCGATAGCTCTGGCAACAAAAAGAGCGGTACCTGGGATTCAGGCACCTCGACGCTGACC
ATTTCTAAAAATCGTACGAAAACCAAGCAGCTGGTCTTCACGAAAGAAGATACGATCACCG
TGCAAAACTATGACAGCGCAGGTACCAATCTGGAAGGCAAAGCTGTGGAAATTACCACGCT
GAAAGAACTGTGTAATGCTCTGAAAGGTACTAGTGACAAAAACAATGGCTCTGGTAGCAAA
GAGAAAAACAAAGATGGCAAGTACTCATTCAACGGCAAAGGTGAAACGAGCGAAAAGACCA
TCGTGCGTGCGAACGGTACCCGCCTGGAATATACGGACATTAAATCGGACGGCAGCGGCAA
AGCAAAGGAAGTCCTGAAAGATTTTACGCTGGAAGGTACCCTGGCAGCAGACGGTAAAACC
ACGCTGAAGGTGACGTGCGGCACCGTGGTTCTGTCAAAAAACATTCTGAAGTCGGGTGAAA
TCACCGCAGCTCTGGATGACAGCGATACCACGCGTGCTACGAAAAAGACCGGTAAATGGGA
TAGCAAGACCTCTACGCTGACCATTAGTGTCAACTCCCAGAAAACGAAGAATCTGGTGTTC
ACCAAAGAAGATACGATCACCGTTCAACGCTATGACAGTGCGGGCACCAACCTGGAAGGCA
AAGCCGTTGAAATTACCACGCTGAAAGAACTGTGTAATGCTCTGAAA

Further information on the heterodimers and their production is derivable from WO 2015/104396 A1, wherein Lip-S1D1-S2D1, Lip-S4D1-S3hybD1 and Lip-S5D1-S6D1 correspond to SEQ ID NOs: 29, 27 and 33, respectively.

In a preferred embodiment of the present invention, the protein is an immunogenic variant with a sequence identity of 60%, 70%, 80%, 85%, 90%, 95%, 96%, 97%, 98%, 99% to SEQ ID NO: 1, 2 or 3.

Sequence identity is frequently measured in terms of percentage identity: the higher the percentage, the more identical the two sequences are. Homologs, orthologs, or variants of a polypeptide will possess a relatively high degree of sequence identity when aligned using standard methods. Methods of alignment of sequences for comparison are well known in the art. Various programs and alignment algorithms are described in: Smith & Waterman (Adv. Appl. Math. 2:482, 1981); Needleman & Wunsch (Mol. Biol. 48:443, 1970); Pearson & Lipman (Proc. Natl. Acad. Sci. USA 85:2444, 1988); Higgins & Sharp (Gene, 73:237-44, 1988); Higgins & Sharp (CABIOS 5: 151-3, 1989); Corpet et al. (Nuc. Acids Res. 16: 10881-90, 1988); Huang et al. (Computer Appls in the Biosciences 8: 155-65, 1992); Pearson et al. (Meth. Mol. Bio. 24:307-31, 1994) and Altschul et al. (J. Mol. Biol. 215:403-10, 1990), presents a detailed consideration of sequence alignment methods and homology calculations. Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. Preferably, the percentage sequence identity is determined over the full length of the sequence. For example, a peptide sequence that has 1166 matches when aligned with a test sequence having 1554 amino acids is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

The NCBI Basic Local Alignment Search Tool (BLAST) (Altschul et al. 1990. Mol. Biol. 215:403) is available from several sources, including the National Center for Biotechnology Information (NCBI, Bethesda, MD) and on the internet, for use in connection with the sequence analysis programs BLASTP, BLASTN, BLASTX, TBLASTN and TBLASTX. A description of how to determine sequence identity using this program is available on the NCBI website on the internet. The BLAST and the BLAST 2.0 algorithm are also described in Altschul et al. (Nucleic Acids Res. 25: 3389-3402, 1977). Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff 1992. Proc. Natl. Acad. Sci. USA 89: 10915-10919).

Variants of a protein are typically characterized by possession of at least about 60%, for example at least about 70%, 75%, 80%, 85%, 90%, 91%, 92%, 93%, 94%, 95%, 96%, 97%, 98% or 99% sequence identity counted over at least defined number of amino acid residues of the reference sequence, over the full length of the reference sequence or over the full length alignment with the reference amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used.

One example of a useful algorithm is PILEUP. PILEUP uses a simplification of the progressive alignment method of Feng & Doolittle {Mol. Evol. 35: 351-360, 1987). The method used is similar to the method described by Higgins & Sharp (CABIOS 5: 151-153, 1989). Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al. 1984. Nuc. Acids Res. 12: 387-395).

As used herein, reference to “at least 80% identity” refers to at least 80%, at least 85%, at least 90%, at least 91%, at least 92%, at least 93%, at least 94%, at least 95%, at least 96%, at least 97%, at least 98%, at least 99%, or even 100% identity to a specified reference sequence, e.g. to at least 50, 100, 150, 250, 500 amino acid residues of the reference sequence or to the full length of the sequence. As used herein, reference to “at least 90% identity” refers to at least 90%, at least 91%, at least 92%, at least 93%, at least 94%, at least 95%, at least 96%, at least 97%, at least 98%, at least 99%, or even 100% identity to a specified reference sequence, e.g. to at least 50, 100, 150, 250, 500 amino acid residues of the reference sequence or to the full length of the sequence.

An immunogenic variant can induce neutralizing antibodies recognizing the native protein of the pathogen in question.

Also preferably, the protein in the vaccine is a Clostridium difficile toxin protein, particularly a Clostridium difficile toxin fusion protein. The Clostridium difficile toxin fusion protein comprises parts of toxin A fused to toxin B, particularly a part of the C-terminal domain of toxin A fused to a part of the C-terminal domain of toxin B.

Most preferably, the vaccine comprises Clostridium difficile toxin fusion protein of SEQ ID NO: 7 (C-TAB.G5) and/or Clostridium difficile toxin fusion protein of SEQ ID NO: 8 (C-TAB.G5.1).

The C-TAB.G5 or C-TAB.G5.1 comprises 19 repeating units of the C-terminal domain of toxin A fused to 23 repeating units of the C-terminal domain of toxin B. The present invention also includes compositions and formulations comprising the C-TAB.G5 or C-TAB.G5.1 isolated polypeptide. The sequences are shown as following SEQ ID NO: 7 and 8:

C-TAB.G5-aa
SEQ ID NO: 7
MVTGVFKGPNGFEYFAPANTHNNNIEGQAIVYQNKFLTLNGKKYYFDNDSKAVTGWQTIDG
KKYYFNLNTAEAATGWQTIDGKKYYFNLNTAEAATGWQTIDGKKYYFNTNTFIASTGYTSI
NGKHFYFNTDGIMQIGVFKGPNGFEYFAPANTHNNNIEGQAILYQNKFLTLNGKKYYFGSD
SKAVTGLRTIDGKKYYFNTNTAVAVTGWQTINGKKYYFNTNTSIASTGYTIISGKHFYFNT
DGIMQIGVFKGPDGFEYFAPANTDANNIEGQAIRYQNRFLYLHDNIYYFGNNSKAATGWVT
IDGNRYYFEPNTAMGANGYKTIDNKNFYFRNGLPQIGVFKGSNGFEYFAPANTDANNIEGQ
AIRYQNRFLHLLGKIYYFGNNSKAVTGWQTINGKVYYFMPDTAMAAAGGLFEIDGVIYFFG
VDGVKAPGIYGRSMHNLITGFVTVGDDKYYFNPINGGAASIGETIIDDKNYYFNQSGVLQT
GVFSTEDGFKYFAPANTLDENLEGEAIDFTGKLIIDENIYYFDDNYRGAVEWKELDGEMHY
FSPETGKAFKGLNQIGDYKYYFNSDGVMQKGFVSINDNKHYFDDSGVMKVGYTEIDGKHFY
FAENGEMQIGVENTEDGFKYFAHHNEDLGNEEGEEISYSGILNFNNKIYYFDDSFTAVVGW
KDLEDGSKYYFDEDTAEAYIGLSLINDGQYYFNDDGIMQVGFVTINDKVFYFSDSGIIESG
VQNIDDNYFYIDDNGIVQIGVFDTSDGYKYFAPANTVNDNIYGQAVEYSGLVRVGEDVYYF
GETYTIETGWIYDMENESDKYYFNPETKKACKGINLIDDIKYYFDEKGIMRTGLISFENNN
YYFNENGEMQFGYINIEDKMFYFGEDGVMQIGVENTPDGFKYFAHQNTLDENFEGESINYT
GWLDLDEKRYYFTDEYIAATGSVIIDGEEYYFDPDTAQLVISE
C-TAB.G5.1-aa
SEQ ID NO: 8
VTGVFKGPNGFEYFAPANTHNNNIEGQAIVYQNKFLTLNGKKYYFDNDSKAVTGWQTIDGK
KYYFNLNTAEAATGWQTIDGKKYYFNLNTAEAATGWQTIDGKKYYFNTNTFIASTGYTSIN
GKHFYFNTDGIMQIGVFKGPNGFEYFAPANTDANNIEGQAILYQNKFLTLNGKKYYFGSDS
KAVTGLRTIDGKKYYFNTNTAVAVTGWQTINGKKYYFNTNTSIASTGYTIISGKHFYFNTD
GIMQIGVFKGPDGFEYFAPANTDANNIEGQAIRYQNRFLYLHDNIYYFGNNSKAATGWVTI
DGNRYYFEPNTAMGANGYKTIDNKNFYFRNGLPQIGVFKGSNGFEYFAPANTDANNIEGQA
IRYQNRFLHLLGKIYYFGNNSKAVTGWQTINGKVYYFMPDTAMAAAGGLFEIDGVIYFFGV
DGVKAPGIYGRSMHNLITGFVTVGDDKYYFNPINGGAASIGETIIDDKNYYFNQSGVLQTG
VFSTEDGFKYFAPANTLDENLEGEAIDFTGKLIIDENIYYFDDNYRGAVEWKELDGEMHYF
SPETGKAFKGLNQIGDYKYYFNSDGVMQKGFVSINDNKHYFDDSGVMKVGYTEIDGKHFYF
AENGEMQIGVENTEDGFKYFAHHNEDLGNEEGEEISYSGILNENNKIYYFDDSFTAVVGWK
DLEDGSKYYFDEDTAEAYIGLSLINDGQYYFNDDGIMQVGFVTINDKVFYFSDSGIIESGV
QNIDDNYFYIDDNGIVQIGVFDTSDGYKYFAPANTVNDNIYGQAVEYSGLVRVGEDVYYFG
ETYTIETGWIYDMENESDKYYFNPETKKACKGINLIDDIKYYFDEKGIMRTGLISFENNNY
YFNENGEMQFGYINIEDKMFYFGEDGVMQIGVENTPDGFKYFAHQNTLDENFEGESINYTG
WLDLDEKRYYFTDEYIAATGSVIIDGEEYYFDPDTAQLVISE

The nucleic acid sequences encoding the above proteins are as follows:

C-TAB.G5-nt
SEQ ID NO: 9
ATGCATAATTTGATAACTGGATTTGTGACTGTAGGCGATGATAAATACTACTTTAATCCAA
TTAATGGTGGAGCTGCTTCAATTGGAGAGACAATAATTGATGACAAAAATTATTATTTCAA
CCAAAGTGGAGTGTTACAAACAGGTGTATTTAGTACAGAAGATGGATTTAAATATTTTGCC
CCAGCTAATACACTTGATGAAAACCTAGAAGGAGAAGCAATTGATTTTACTGGAAAATTAA
TTATTGACGAAAATATTTATTATTTTGATGATAATTATAGAGGAGCTGTAGAATGGAAAGA
ATTAGATGGTGAAATGCACTATTTTAGCCCAGAAACAGGTAAAGCTTTTAAAGGTCTAAAT
CAAATAGGTGATTATAAATACTATTTCAATTCTGATGGAGTTATGCAAAAAGGATTTGTTA
GTATAAATGATAATAAACACTATTTTGATGATTCTGGTGTTATGAAAGTAGGTTACACTGA
AATAGATGGCAAGCATTTCTACTTTGCTGAAAACGGAGAAATGCAAATAGGAGTATTTAAT
ACAGAAGATGGATTTAAATATTTTGCTCATCATAATGAAGATTTAGGAAATGAAGAAGGTG
AAGAAATCTCATATTCTGGTATATTAAATTTCAATAATAAAATTTACTATTTTGATGATTC
ATTTACAGCTGTAGTTGGATGGAAAGATTTAGAGGATGGTTCAAAGTATTATTTTGATGAA
GATACAGCAGAAGCATATATAGGTTTGTCATTAATAAATGATGGTCAATATTATTTTAATG
ATGATGGAATTATGCAAGTTGGATTTGTCACTATAAATGATAAAGTCTTCTACTTCTCTGA
CTCTGGAATTATAGAATCTGGAGTACAAAACATAGATGACAATTATTTCTATATAGATGAT
AATGGTATAGTTCAAATTGGTGTATTTGATACTTCAGATGGATATAAATATTTTGCACCTG
CTAATACTGTAAATGATAATATTTACGGACAAGCAGTTGAATATAGTGGTTTAGTTAGAGT
TGGGGAAGATGTATATTATTTTGGAGAAACATATACAATTGAGACTGGATGGATATATGAT
ATGGAAAATGAAAGTGATAAATATTATTTCAATCCAGAAACTAAAAAAGCATGCAAAGGTA
TTAATTTAATTGATGATATAAAATATTATTTTGATGAGAAGGGCATAATGAGAACGGGTCT
TATATCATTTGAAAATAATAATTATTACTTTAATGAGAATGGTGAAATGCAATTTGGTTAT
ATAAATATAGAAGATAAGATGTTCTATTTTGGTGAAGATGGTGTCATGCAGATTGGAGTAT
TTAATACACCAGATGGATTTAAATACTTTGCACATCAAAATACTTTGGATGAGAATTTTGA
GGGAGAATCAATAAACTATACTGGTTGGTTAGATTTAGATGAAAAGAGATATTATTTTACA
GATGAATATATTGCAGCAACTGGTTCAGTTATTATTGATGGTGAGGAGTATTATTTTGATC
CTGATACAGCTCAATTAGTGATTAGTGAATAG
C-TAB.G5.1-nt
SEQ ID NO: 10
CCATGGTTACAGGTGTTTTCAAAGGTCCGAACGGCTTTGAATATTTTGCACCGGCAAATAC
CCACAATAATAATATTGAAGGCCAGGCCATCGTGTATCAGAATAAATTTCTGACCCTGAAC
GGCAAAAAATACTATTTCGATAACGATAGCAAAGCAGTTACCGGTTGGCAAACCATTGATG
GCAAAAAATATTACTTCAACCTGAATACCGCAGAAGCAGCAACCGGCTGGCAGACGATCGA
CGGTAAAAAGTACTATTTTAACCTGAACACAGCCGAAGCCGCTACAGGCTGGCAGACAATA
GATGGGAAGAAGTATTATTTTAATACCAATACCTTTATTGCCAGCACCGGCTATACCAGCA
TTAATGGCAAACACTTCTATTTTAACACCGATGGTATTATGCAGATCGGTGTGTTTAAGGG
CCCTAATGGTTTTGAGTACTTCGCTCCGGCTAATACCGATGCAAATAACATCGAAGGTCAG
GCAATTCTGTACCAGAACAAATTTTTAACGCTGAACGGTAAGAAATATTACTTTGGTAGCG
ATTCAAAAGCCGTTACCGGTCTGCGTACGATCGACGGCAAGAAATATTATTTCAATACAAA
CACCGCAGTTGCCGTGACAGGTTGGCAGACGATAAATGGTAAGAAGTACTACTTCAACACC
AATACCAGCATTGCAAGTACCGGTTATACCATTATCAGCGGCAAACACTTTTACTTCAATA
CAGACGGCATTATGCAGATTGGCGTTTTCAAAGGTCCGGATGGTTTCGAGTACTTTGCCCC
TGCAAATACAGATGCAAACAATATTGAGGGACAGGCAATTCGCTATCAGAATCGTTTTCTG
TATCTGCACGATAACATCTATTACTTCGGCAATAATTCAAAAGCAGCCACCGGTTGGGTTA
CAATTGATGGTAATCGTTATTACTTTGAGCCGAATACCGCAATGGGTGCAAATGGTTATAA
AACCATCGATAACAAAAATTTTTATTTCCGCAACGGTCTGCCGCAGATTGGTGTTTTTAAG
GGTAGCAATGGCTTCGAGTATTTTGCGCCAGCCAACACCGATGCCAACAACATTGAAGGCC
AAGCGATTCGTTATCAAAACCGCTTTCTGCATCTGCTGGGCAAAATTTATTACTTTGGCAA
CAATAGCAAAGCGGTGACGGGCTGGCAAACCATTAACGGTAAAGTTTATTATTTCATGCCG
GATACCGCTATGGCAGCAGCCGGTGGTCTGTTTGAAATTGATGGCGTGATTTATTTTTTTG
GCGTGGATGGTGTTAAAGCACCGGGTATTTATGGTCGTAGCATGCATAATCTGATTACCGG
TTTTGTTACCGTGGGCGACGATAAATACTACTTTAATCCGATTAATGGTGGTGCAGCAAGC
ATTGGTGAAACCATTATCGATGACAAAAACTATTATTTTAACCAGAGCGGTGTTCTGCAGA
CAGGTGTTTTTAGCACCGAAGATGGCTTCAAATATTTTGCTCCTGCGAATACACTGGATGA
AAATCTGGAAGGTGAAGCAATTGATTTTACCGGCAAACTGATCATCGACGAGAACATCTAC
TATTTTGATGATAATTATCGCGGTGCCGTGGAATGGAAAGAACTGGATGGTGAAATGCACT
ATTTTAGTCCGGAAACCGGTAAAGCCTTTAAAGGTCTGAATCAGATCGGCGATTACAAGTA
TTACTTTAATTCAGATGGCGTGATGCAGAAAGGCTTTGTGAGCATTAACGACAACAAACAC
TATTTTGACGACAGCGGTGTGATGAAAGTGGGTTATACCGAAATCGACGGGAAACATTTTT
ATTTTGCCGAAAACGGCGAAATGCAGATTGGAGTATTTAATACCGAGGACGGCTTTAAATA
CTTTGCCCATCATAATGAAGATCTGGGTAATGAAGAAGGCGAAGAAATTAGCTATAGCGGC
ATTCTGAATTTTAATAACAAGATCTATTATTTCGATGATAGCTTCACCGCAGTTGTTGGTT
GGAAAGATCTGGAAGATGGCAGCAAATATTATTTTGATGAAGATACCGCAGAGGCCTATAT
TGGTCTGAGCCTGATTAATGATGGCCAGTATTATTTCAACGATGATGGTATCATGCAGGTT
GGTTTTGTGACCATCAACGATAAAGTGTTCTATTTCAGCGATAGCGGCATTATTGAAAGCG
GTGTTCAGAACATCGACGATAACTATTTCTACATCGATGATAACGGTATTGTTCAGATTGG
CGTGTTTGATACCTCCGATGGTTATAAATATTTCGCACCAGCCAATACCGTGAACGATAAT
ATTTATGGTCAGGCAGTTGAATATTCAGGTCTGGTTCGTGTTGGCGAAGATGTTTATTATT
TTGGCGAAACCTATACCATTGAAACCGGCTGGATCTATGATATGGAAAACGAGAGCGACAA
GTACTATTTCAATCCGGAAACGAAAAAAGCCTGCAAAGGCATTAATCTGATCGACGATATT
AAGTACTACTTTGACGAAAAAGGCATTATGCGTACCGGTCTGATTAGCTTTGAGAACAACA
ACTATTACTTCAATGAGAACGGTGAGATGCAGTTTGGCTATATCAACATCGAGGACAAAAT
GTTTTATTTTGGTGAGGACGGTGTGATGCAGATAGGGGTTTTTAATACACCGGATGGGTTT
AAGTATTTTGCACATCAGAACACCCTGGATGAAAACTTTGAAGGCGAAAGCATTAATTATA
CCGGTTGGCTGGATCTGGATGAGAAACGTTATTATTTCACCGACGAATACATTGCAGCAAC
CGGTAGCGTTATTATTGATGGTGAGGAATATTACTTCGATCCGGATACAGCACAGCTGGTT
ATTAGCGAATAACTCGAG

Further information on the proteins C-TAB.G5 and C-TAB.G5.1 and their production is derivable from WO 2012028741 A1 and EP2753352 B2, wherein C-TAB.G5 and C-TAB.G5.1 correspond to SEQ ID NOs: 2 and 4, respectively.

In a preferred embodiment, the protein is an immunogenic variant with a sequence identity of 60%, 70%, 80%, 85%, 90%, 95%, 96%, 97%, 98%, 99% to SEQ ID NOs: 7, 8 or 12.

In another preferred embodiment of the present invention, the lipidated protein is a lipidated SARS-CoV-2 spike protein, particularly a lipidated form of a protein comprising the protein of SEQ ID NO: 15 or an immunogenic variant thereof with a sequence identity of 60%, 70%, 80%, 85%, 90%, 95%, 96%, 97%, 98%, 99% to SEQ ID NO: 15.

LIP-Protein\S_2019-nCoV/Italy-INMI1
(Sprotein_hCoV19ItalyINMI1is|2020)
SEQ ID NO: 15
LipCSSMFVFLVLLPLVSSQCVNLTTRTQLPPAYTNSFTRGVYYPDKVFRSSVLHSTQDLF
LPFFSNVTWFHAIHVSGTNGTKRFDNPVLPENDGVYFASTEKSNIIRGWIFGTTLDSKTQS
LLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRF
QTLLALHRSYLTPGDSSSGWTAGAAAYYVGYLQPRTFLLKYNENGTITDAVDCALDPLSET
KCTLKSFTVEKGIYQTSNFRVQPTESIVRFPNITNLCPFGEVENATRFASVYAWNRKRISN
CVADYSVLYNSASFSTFKCYGVSPTKLNDLCFTNVYADSFVIRGDEVRQIAPGQTGKIADY
NYKLPDDFTGCVIAWNSNNLDSKVGGNYNYLYRLFRKSNLKPFERDISTEIYQAGSTPCNG
VEGFNCYFPLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNT
SNQVAVLYQDVNCTEVPVAIHADQLTPTWRVYSTGSNVFQTRAGCLIGAEHVNNSYECDIP
IGAGICASYQTQTNSPRRARSVASQSIIAYTMSLGAENSVAYSNNSIAIPTNFTISVTTEI
LPVSMTKTSVDCTMYICGDSTECSNLLLQYGSFCTQLNRALTGIAVEQDKNTQEVFAQVKQ
IYKTPPIKDFGGFNFSQILPDPSKPSKRSFIEDLLFNKVTLADAGFIKQYGDCLGDIAARD
LICAQKFNGLTVLPPLLTDEMIAQYTSALLAGTITSGWTFGAGAALQIPFAMQMAYRENGI
GVTQNVLYENQKLIANQFNSAIGKIQDSLSSTASALGKLQDVVNQNAQALNTLVKQLSSNF
GAISSVLNDILSRLDKVEAEVQIDRLITGRLQSLQTYVTQQLIRAAEIRASANLAATKMSE
CVLGQSKRVDFCGKGYHLMSFPQSAPHGVVFLHVTYVPAQEKNFTTAPAICHDGKAHFPRE
GVFVSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIKWPW
YIWLGFIAGLIAIVMVTIMLCCMTSCCSCLKGCCSCGSCCKFDEDDSEPVLKGVKLHYT

In another preferred embodiment of the present invention, the protein in the vaccine is an hMPV protein, particularly an hMPV F protein, especially in lipidated form, or an immunogenic variant thereof with a sequence identity of 60%, 70%, 80%, 85%, 90%, 95%, 96%, 97%, 98%, 99% to SEQ ID NOs: 16-22. Further information on the hMPV protein is derivable from WO 2020/234300 A1.

L7F_A1_23 protein sequence
SEQ ID NO: 16
MSWKVVIIFSLLITPQHGLKESYLEESCSTITEGYLSVLRTGWYTNVFTLEVGDVENLTCA
DGPSLIKTELDLTKSALRELRTVSADQLAREEQIEQPRQSGCGAGATAGVAIAKTIRLESE
VTAIKNALKKTNEAVSTLGNGVRVLATAVRELKDFVSKNLTRAINKNKCDIADLKMAVSFS
QFNRRFLNVVRQFSDNAGITPAISLDLMTDAELARAVSNMPTSAGQIKLMLENRAMVRRKG
FGFLIGVYGSSVIYMVQLPIFGVIDTPCWIVKAAPSCSEKKGNYACLLREDQGWYCQNAGS
TVYYPNEKDCETRGDHVFCDTCAGINVAEQSKECNINISTTNYPCKVSTGRHPISMVALSP
LGALVACYKGVSCSIGSNRVGIIKQLNKGCSYITNQDADTVTIDNTVYQLSKVEGEQHVIK
GRPVSSSFDPVKFPEDQFNVALDQVFESIENSQALVDQSNRILSAGYIPEAPRDGQAYVRK
DGEWVLLSTFLGGLVPRGSHHHHHHSAWSHPQFEK
sF_A1_K_L7 protein sequence
SEQ ID NO: 17
MSWKVVIIFSLLITPQHGLKESYLEESCSTITEGYLSVLRTGWYTNVFTLEVGDVENLTCA
DGPSLIKTELDLTKSALRELRTVSADQLAREEQIEQPRQSGCGAGATAGVAIAKTIRLESE
VTAIKNALKKTNEAVSTLGNGVRVLAFAVRELKDFVSKNLTRALNKNKCDIADLKMAVSFS
QFNRRFLNVVRQFSDNAGITPAISLDLMTDAELARAVSNMPTSAGQIKLMLENRAMVRRKG
FGFLIGVYGSSVIYMVQLPIFGVIDTPCWIVKAAPSCSEKKGNYACLLREDQGWYCQNAGS
TVYYPNEKDCETRGDHVFCDTCAGINVAEQSKECNINISTTNYPCKVSTGRHPISMVALSP
LGALVACYKGVSCSIGSNRVGIIKQLNKGCSYITNQDADTVTIDNTVYQLSKVEGEQHVIK
GRPVSSSFDPVKFPEDQFNVALDQVFESIENSQALVDQSNRILSSAESAIGGYIPEAPRDG
QAYVRKDGEWVLLSTFLGGLVPRGSHHHHHHSAWSHPQFEK
L7F_A1_31 protein sequence
SEQ ID NO: 18
MSWKVVIIFSLLITPQHGLKESYLEESCSTITEGYLSVLRTGWYTNVFMLEVGDVENLTCA
DGPSLLKTELDLTKSALRNLRTVSADQLAREEQIEQPRQSGCGAGATAGVAIAKTIRLESE
VTAIKNALKKTNEAVSTLGNGVRVLATMVRELKDFVSKNLTRAINKNKCDIADLKMAVSFS
QFNRRFLNVVRQFSDNAGITPAISLDLMTDAELARAVSNMPTSAGQIKLMLENRAMVRRKG
FGILIGVYGSSVIYMVQLPIFGVIDTPCWIVKAAPSCSEKKGNYACLLREDQGWYCQNAGS
TVYYPNEKDCETRGDHVFCDTCAGINVAEQSKECNINISTTNYPCKVSTGRHPISMVALSP
LGALVACYKGVSCSIGSNRVGIIKQLNKGCSYITNQDADTVTIDNTVYQLSKVEGEQHVIK
GRPVSSSFDPVKFPEDQFNVALDQVFESIENSQALVDQSNRILSAGYIPEAPRDGQAYVRK
DGEWVLLSTFLGGLVPRGSHHHHHHSAWSHPQFEK
L7F_A1_33 protein sequence
SEQ ID NO: 19
MSWKVVIIFSLLITPQHGLKESYLEESCSTITEGYLSVLRTGWYTNVFMLCVGDVENLTCA
DGPSLLKTELDLTKSALRELRTVSADQLAREEQIEQPRQSGCGAGATAGVAIAKTIRLESE
VTAIKNALKKTNEAVSTLGNGVRVLATMVRELCDFVSKNLTRAINKNKCDIADLKMAVSFS
QFNRRFLNVVRQFSDNAGITPAISLDLMTDAELARAVSNMPTSAGQIKLMLENRAMVRRKG
FGFLIGVYGSDVIYMVQLPIFGVIDTPCWIVKAAPSCSEKKGNYACLLREDQGWYCQNAGS
TVYYPNEKDCETRGDHVFCDTCAGINVAEQSKECNINISTTNYPCKVSTGRHPISMVALSP
LGALVACYKGVSCSIGSNRVGIIKQLNKGCSYITNQDADTVTIDNTVYQLSKVEGEQHVIK
GRPVSSSFDPVKFPEDQFNVALDQVFESIENSQALVDQSNRCCSAGYIPEAPRDGQAYVRK
DGEWVLLSTFLGGLVPRGSHHHHHHSAWSHPQFEK
L7F_A1_4.2 protein sequence
SEQ ID NO: 20
MSWKVVIIFSLLITPQHGLKESYLEESCSTITEGYLSVLRTGWYTNVFMLEVGDVENLTCA
DGPSLIKTELDLTKSALRELRTVSADQLAREEQIEQPRQSGCGAGATAGVAIAKTIRLESE
VTAWKNALKKTNEVVSTLGNGVRVLVTMVRELKDFVSKNLTRALNKNKCDIADLKMAVSFS
QFNRRFLNVVRQFSDNAGITPAISLDLMTDAELARAVSNMPTSAGQIKLMLENRAMVRRKG
FGFLIGVYGSSVIYMVQLPIFGVIDTPCWIVKAAPSCSEKKGNYACLLREDQGWYCQNAGS
TVYYPNEKDCETRGDHVFCDTCAGINVAEQSKECNINISTTNYPCKVSTGRHPISMVALSP
LGALVACYKGVSCSIGSNRVGIIKQLNKGCSYITNQDADTVTIDNTVYQLSKVEGEQHVIK
GRPVSSSFDPVKFPEDQFNVALDQVFESIENSQALVDQSNRILSSAESAIGGYIPEAPRDG
QAYVRKDGEWVLLSTFLGGLVPRGSHHHHHHSAWSHPQFEK
sF_A1_K-E294 protein sequence
with substitutions A113C, A339C, T160F, 1177L and trimer-
ization helper KLL
SEQ ID NO: 21
MSWKVVIIFSLLITPQHGLKESYLEESCSTITEGYLSVLRTGWYTNVFTLEVGDVENLTCA
DGPSLIKTELDLTKSALRELRTVSADQLAREEQIENPRQSRFVLGAIALGVCTAAAVTAGV
AIAKTIRLESEVTAIKNALKKTNEAVSTLGNGVRVLAFAVRELKDFVSKNLTRALNKNKCD
IADLKMAVSFSQFNRRFLNVVRQFSDNAGITPAISLDLMTDAELARAVSNMPTSAGQIKLM
LENRAMVRRKGFGFLIGVYGSSVIYMVQLPIFGVIDTPCWIVKAAPSCSEKKGNYACLLRE
DQGWYCQNAGSTVYYPNEKDCETRGDHVFCDTACGINVAEQSKECNINISTTNYPCKVSTG
RHPISMVALSPLGALVACYKGVSCSIGSNRVGIIKQLNKGCSYITNQDADTVTIDNTVYQL
SKVEGEQHVIKGRPVSSSFDPVKFPEDQFNVALDQVFESIENSQALVDQSNRILSSAESAI
GGYIPEAPRDGQAYVRKDGEWVLLSTFLGGLVPRGSHHHHHHSAWSHPQFEK
SF_A1_MFur protein sequence
with deletion of amino acids at positions 103 to 111,
replacement of R102 by a furin site KKRKRR and the
substitution G294E, stabilized in post-fusion conformation
SEQ ID NO: 22
MSWKVVIIFSLLITPQHGLKESYLEESCSTITEGYLSVLRTGWYTNVFTLEVGDVENLTCA
DGPSLIKTELDLTKSALRELRTVSADQLAREEQIENPRQSKKRKRRVATAAAVTAGVAIAK
TIRLESEVTAIKNALKKTNEAVSTLGNGVRVLATAVRELKDFVSKNLTRAINKNKCDIADL
KMAVSFSQFNRRFLNVVRQFSDNAGITPAISLDLMTDAELARAVSNMPTSAGQIKLMLENR
AMVRRKGFGFLIGVYGSSVIYMVQLPIFGVIDTPCWIVKAAPSCSEKKGNYACLLREDQGW
YCQNAGSTVYYPNEKDCETRGDHVFCDTAAGINVAEQSKECNINISTTNYPCKVSTGRHPI
SMVALSPLGALVACYKGVSCSIGSNRVGIIKQLNKGCSYITNQDADTVTIDNTVYQLSKVE
GEQHVIKGRPVSSSFDPVKFPEDQFNVALDQVFESIENSQALVDQSNRILSSAEKGNTSGR
ENLYFQGGGGSGYIPEAPRDGQAYVRKDGEWVLLSTFLGGIEGRHHHHHH

Typically, the copper as impurity in the aluminum composition for use of the present invention is in form of an ion, particularly as Cu⁺ and/or Cu²⁺.

As detailed above, a radical quenching compound, preferably L-methionine, may be used to prevent excess copper from reducing bioavailability in the aluminum composition for use in the present invention. A typical suitable concentration of L-methionine in the composition is at least 10 mmol/l. Depending on the amount of copper in the composition, the concentration of the radical quenching compound may be even higher such as at least 20 mmol/l, at least 30 mmol/l, at least 40 mmol/1 or at least 50 mmol/1 or lower such as at most 10 mmol/l, at most 5 mmol/1 or at most 1 mmol/l.

Alternatively, the concentration of L-methionine is determined based on the concentration of copper in the composition. Particularly, the concentration of the radical quenching compound, preferably L-methionine, in mol/l, is at least equivalent to the concentration of copper in the composition. Alternatively, the concentration of L-methionine in mol/l is at least twice, threefold, fourfold, fivefold, or even tenfold, the concentration of copper in the composition.

In one embodiment, the composition for use further comprises a reactive compound, wherein the reactive compound is selected from the group consisting of a redox active compound, a radical building compound, a stabilizing compound and a combination of any thereof. Without being bound to the theory, antigen degradation of protein vaccines in aqueous compositions comprising heavy metal ions present in an aluminium salt, such as aluminium hydroxide, might be explained with an underlying degradation pathway involving free radicals such as e.g. free radicals of sulfite. Heavy metal-catalyzed oxidation is a degradation pathway resulting in the covalent modification of proteins. The modified physicochemical properties of the oxidized/modified protein or antigen may result in loss of biological activity. Redox active compounds are suitable to prevent this modification. Preferably, the reactive compound is selected from the group consisting of formaldehyde, ethanol, chloroform, trichloroethylene, acetone, triton-X-100, deoxycholate, diethylpyrocarbonate, sulfite, Na₂S₂O₅, beta-propiolactone, polysorbate such as Tween 20®, Tween 80®, O₂, phenol, pluronic type copolymers, and a combination of any thereof.

In one embodiment, the composition comprises 3 heterodimers, preferably Lip-S1D1-S2D1 (SEQ ID NO: 1), Lip-S4D1-S3hybD1 (SEQ ID NO: 2) and Lip-S5D1-S6D1 (SEQ ID NO: 3) at a weight ratio of 1:2:1, 1:3:1, 1:1:2, 1:1:3, 1:2:2, 1:2:3, 1:3:2, 1:3:3, 2:1:1, 2:1:2, 2:1:3, 2:2:3, 2:2:1, 2:3:1, 2:3:2, 2:3:3, 3:1:1, 3:1:2, 3:1:3, 3:2:1, 3:2:2, 3:2:3, 3:3:1, or 3:3:2. Preferably, the above compositions for use according to the present invention are characterized in that the composition comprises the heterodimer proteins in a weight ratio of 1:1:1 (Lip-S1D1-S2D1:Lip-S4D1-S3hybD1:Lip-S5D1-S6D1).

In a preferred embodiment, the OspA vaccine comprises the heterodimer protein of SEQ ID NO: 1 (Lip-S1D1-S2D1), the heterodimer protein of SEQ ID NO: 2 (Lip-S4D1-S3hybD1) and the heterodimer protein of SEQ ID NO: 3 (Lip-S5D1-S6D1), particularly wherein the composition comprises the heterodimer proteins in a weight ratio of 1:1:1 (Lip-S1D1-S2D1:Lip-S4D1-S3hybD1:Lip-S5D1-S6D1); or

the Clostridium difficile vaccine comprises the Clostridium difficile toxin A protein of SEQ ID NO: 13 (Lip-ToxA-His) and/or the Clostridium difficile toxin B protein of SEQ ID NO: 14 (Lip-ToxB-His), particularly wherein the composition comprises the Clostridium difficile toxin proteins in a weight ratio of 1:1 and in particular also without the His-tag; or

the Clostridium difficile vaccine comprises the Clostridium difficile toxin fusion protein of SEQ ID NO: 7 (C-TAB.G5) and/or the Clostridium difficile toxin fusion protein of SEQ ID NO: 8 (C-TAB.G5.1), each in lipidated or unlipidated form; or

the SARS-CoV-2 vaccine comprises the SARS-CoV-2 spike protein of SEQ ID NO: 15; or the vaccine comprises an immunogenic variant of any of the proteins defined above, particularly wherein the variant has sequence identity to any of the proteins of SEQ ID NOs: 1, 2, 3, 7, 8, 13, 14, or 15 of not less than 80%, 85%, 90%, 95%, 96%, 97%, 98%, or 99%; or

the hMPV vaccine comprises the hMPV F protein of any of SEQ ID NOs: 16-22, each in lipidated or unlipidated form; or

the vaccine comprises an immunogenic variant of any of the proteins defined above, particularly wherein the variant has sequence identity to any of the proteins of SEQ ID NOs: 1, 2, 3, 7, 8, 13, 14, 15, 16, 17, 18, 19, 20, 21, or 22 of not less than 80%, 85%, 90%, 95%, 96%, 97%, 98%, or 99%.

The sequences of SEQ ID NOs: 12, 13 and 14 are as follows:

Lip-C-TAB.G5.1
SEQ ID NO: 12
LipCSSFVTGVFKGPNGFEYFAPANTHNNNIEGQAIVYQNKFLTLNGKKYYFDNDSKAVTG
WQTIDGKKYYFNLNTAEAATGWQTIDGKKYYFNLNTAEAATGWQTIDGKKYYFNTNTFIAS
TGYTSINGKHFYFNTDGIMQIGVFKGPNGFEYFAPANTDANNIEGQAILYQNKELTLNGKK
YYFGSDSKAVTGLRTIDGKKYYFNTNTAVAVTGWQTINGKKYYFNTNTSIASTGYTIISGK
HFYFNTDGIMQIGVFKGPDGFEYFAPANTDANNIEGQAIRYQNRFLYLHDNIYYFGNNSKA
ATGWVTIDGNRYYFEPNTAMGANGYKTIDNKNFYFRNGLPQIGVFKGSNGFEYFAPANTDA
NNIEGQAIRYQNRFLHLLGKIYYFGNNSKAVTGWQTINGKVYYFMPDTAMAAAGGLFEIDG
VIYFFGVDGVKAPGIYGRSMHNLITGFVTVGDDKYYFNPINGGAASIGETIIDDKNYYFNQ
SGVLQTGVFSTEDGFKYFAPANTLDENLEGEAIDFTGKLIIDENIYYFDDNYRGAVEWKEL
DGEMHYFSPETGKAFKGLNQIGDYKYYFNSDGVMQKGFVSINDNKHYFDDSGVMKVGYTEI
DGKHFYFAENGEMQIGVFNTEDGFKYFAHHNEDLGNEEGEEISYSGILNFNNKIYYFDDSF
TAVVGWKDLEDGSKYYFDEDTAEAYIGLSLINDGQYYFNDDGIMQVGFVTINDKVFYFSDS
GIIESGVQNIDDNYFYIDDNGIVQIGVFDTSDGYKYFAPANTVNDNIYGQAVEYSGLVRVG
EDVYYFGETYTIETGWIYDMENESDKYYFNPETKKACKGINLIDDIKYYFDEKGIMRTGLI
SFENNNYYFNENGEMQFGYINIEDKMFYFGEDGVMQIGVENTPDGFKYFAHQNTLDENFEG
ESINYTGWLDLDEKRYYFTDEYIAATGSVIIDGEEYYFDPDTAQLVISE
Lip-ToxA-His
SEQ ID NO: 13
LipCSSFVTGVFKGPNGFEYFAPANTHNNNIEGQAIVYQNKFLTLNGKKYYFDNDSKAVTG
WQTIDGKKYYFNLNTAEAATGWQTIDGKKYYFNLNTAEAATGWQTIDGKKYYFNTNTFIAS
TGYTSINGKHFYFNTDGIMQIGVFKGPNGFEYFAPANTDANNIEGQAILYQNKFLTLNGKK
YYFGSDSKAVTGLRTIDGKKYYFNTNTAVAVTGWQTINGKKYYFNTNTSIASTGYTIISGK
HFYFNTDGIMQIGVFKGPDGFEYFAPANTDANNIEGQAIRYQNRFLYLHDNIYYFGNNSKA
ATGWVTIDGNRYYFEPNTAMGANGYKTIDNKNFYFRNGLPQIGVFKGSNGFEYFAPANTDA
NNIEGQAIRYQNRFLHLLGKIYYFGNNSKAVTGWQTINGKVYYFMPDTAMAAAGGLFEIDG
VIYFFGVDGVKAPGIYGLEHHHHHH
Lip-ToxB-His
SEQ ID NO: 14
LipCSSFNLITGFVTVGDDKYYFNPINGGAASIGETIIDDKNYYFNQSGVLQTGVFSTEDG
FKYFAPANTLDENLEGEAIDFTGKLIIDENIYYFDDNYRGAVEWKELDGEMHYFSPETGKA
FKGLNQIGDYKYYFNSDGVMQKGFVSINDNKHYFDDSGVMKVGYTEIDGKHFYFAENGEMQ
IGVENTEDGFKYFAHHNEDLGNEEGEEISYSGILNENNKIYYFDDSFTAVVGWKDLEDGSK
YYFDEDTAEAYIGLSLINDGQYYFNDDGIMQVGFVTINDKVFYFSDSGIIESGVQNIDDNY
FYIDDNGIVQIGVFDTSDGYKYFAPANTVNDNIYGQAVEYSGLVRVGEDVYYFGETYTIET
GWIYDMENESDKYYFNPETKKACKGINLIDDIKYYFDEKGIMRTGLISFENNNYYFNENGE
MQFGYINIEDKMFYFGEDGVMQIGVENTPDGFKYFAHQNTLDENFEGESINYTGWLDLDEK
RYYFTDEYIAATGSVIIDGEEYYFDPDTAQLVISELEHHHHHH

In a preferred embodiment, the aluminum composition for use according to the invention is characterized in that the vaccine is a OspA protein, preferably the heterodimer protein of SEQ ID NO: 1 (Lip-S1D1-S2D1), the heterodimer protein of SEQ ID NO: 2 (Lip-S4D1-S3hybD1) and the heterodimer protein of SEQ ID NO: 3 (Lip-S5D1-S6D1), to be administered
to a human adult a protein content of said 3 heterodimers in the range of from 120 to 200 μg per dose; or
to a human child a protein content of said 3 heterodimers in the range of from 60 to 100 μg per dose.

In a preferred embodiment, the aluminum composition for use according to the invention is characterized in that the vaccine comprises a Clostridium difficile toxin protein, particularly the Clostridium difficile toxin fusion protein of SEQ ID NO: 7 (C-TAB.G5) and/or the Clostridium difficile toxin fusion protein of SEQ ID NO: 8 (C-TAB.G5.1), to be administered to a human at a dose of from 20 to 200 μg.

In another preferred embodiment, the aluminum composition for use according to the invention is characterized in that the vaccine is comprises the heterodimer protein of SEQ ID NO: 1 (Lip-S1D1-S2D1), the heterodimer protein of SEQ ID NO: 2 (Lip-S4D1-S3hybD1) and the heterodimer protein of SEQ ID NO: 3 (Lip-S5D1-S6D1) and is to be administered
to a human adult at least three times at a total protein content of said 3 heterodimers in the range of from 120 to 200 μg per dose; or
to a human child at least three times at a total protein content of said 3 heterodimers in the range of from 60 to 100 μg per dose.

In another preferred embodiment, the aluminum composition for use according to the invention is characterized in that vaccine comprises the Clostridium difficile toxin fusion protein of SEQ ID NO: 7 (C-TAB.G5) and/or the Clostridium difficile toxin fusion protein of SEQ ID NO: 8 (C-TAB.G5.1) to be administered to a human at least three times at a total protein content of said 2 toxin fusion proteins at a dose of from 20 to 200 μg, particularly at a dose of 20 μg at the first administration, at a dose of 75 μg at a second administration and at a dose of 200 μg at a third administration, wherein the second and third administrations are 7 days and 21 days from the first administration, respectively.

In a third aspect, the present invention relates to the use of a composition comprising less than 1.25 ppb copper for increasing the bioavailability of a protein, such as an OspA protein, in an aluminum-containing vaccine composition.

In a fourth aspect, the present invention relates to the use of a radical quenching compound, particularly L-methionine, for increasing bioavailability of a protein, such as an OspA protein, Clostridium difficile toxin protein, spike protein of SARS-CoV-2 or an hMPV F protein, in a copper- and aluminum-containing vaccine composition.

The above definitions, comments and preferred embodiments made with respect to the first and second aspect of the invention apply also to the third and fourth aspect of the invention.

### Further Embodiments of the Present Invention are as Follows

1. A method for adjuvanting a vaccine comprising administering an aluminum composition comprising less than 1.25 ppb copper or a sufficient amount of radical quenching compound to a human, wherein the aluminum composition increases the bioavailability of an antigen in the vaccine, wherein the antigen is a protein.
2. A method for vaccinating a human, the method comprising administering an aluminum composition comprising less than 1.25 ppb copper or a sufficient amount of a radical quenching compound to a human, wherein the aluminum composition increases the bioavailability of an antigen in the vaccine, wherein the antigen is a protein.
3. The method of embodiment 1 or 2, wherein the protein is an OspA protein or a Clostridium difficile toxin protein or a SARS-CoV-2 protein or an hMPV protein, preferably an OspA heterodimer or a Clostridium difficile toxin fusion protein or a spike protein of SARS-CoV-2 or fragment thereof or an hMPV F protein.
4. The method of any of embodiments 1 to 3, wherein copper is in form of an ion, particularly as Cu⁺ or Cu²⁺.
5. The method of any of embodiments 1 to 4, wherein the radical quenching compound is L-methionine, particularly wherein L-methionine is present in a concentration of at least 10 mmol/l.
6. The method of any of embodiments 1 to 5, wherein the concentration of the radical quenching compound, particularly L-methionine, in mol/l is at least equivalent with the concentration of copper in the composition.
7. The method of any of embodiments 1 to 6 further comprising a reactive compound, wherein the reactive compound is selected from the group consisting of a redox active compound, a radical building compound, a stabilizing compound and a combination of any thereof, especially wherein the reactive compound is selected from the group consisting of formaldehyde, ethanol, chloroform, trichloroethylene, acetone, triton-X-100, triton-X-114, deoxycholate, diethylpyrocarbonate, sulfite, Na₂S₂O₅, beta-propiolactone, polysorbate such as Tween 20®, Tween 80®, O₂, phenol, pluronic type copolymers, and a combination of any thereof.
8. The method of any of embodiments 1 to 7,
   a) wherein the OspA vaccine comprises the heterodimer protein of SEQ ID NO: 1 (Lip-S1D1-S2D1), the heterodimer protein of SEQ ID NO: 2 (Lip-S4D1-S3hybD1) and the heterodimer protein of SEQ ID NO: 3 (Lip-S5D1-S6D1), particularly wherein the composition comprises the heterodimer proteins in a weight ratio of 1:1:1 (Lip-S1D1-S2D1:Lip-S4D1-S3hybD1:Lip-S5D1-S6D1); or
   b) wherein the Clostridium difficile vaccine comprises the Clostridium difficile toxin A protein of SEQ ID NO: 13 (Lip-ToxA-His) and/or the Clostridium difficile toxin B protein of SEQ ID NO: 14 (Lip-ToxB-His), particularly wherein the composition comprises the Clostridium difficile toxin proteins in a weight ratio of 1:1; or
   c) wherein the Clostridium difficile vaccine comprises the Clostridium difficile toxin fusion protein of SEQ ID NO: 7 (C-TAB.G5) and/or the Clostridium difficile toxin fusion protein of SEQ ID NO: 8 (C-TAB.G5.1); or
   d) wherein the SARS-CoV-2 vaccine comprises the SARS-CoV-2 spike protein of SEQ ID NO: 15; or
   e) wherein the hMPV vaccine comprises the hMPV F protein of any of SEQ ID NOs: 16-22; or
   f) wherein the vaccine comprises an immunogenic variant of any of the proteins defined in a) to e), particularly wherein the variant has sequence identity to any of the proteins of SEQ ID NOs: 1, 2, 3, 7, 8, 13, 14, 15, 16, 17, 18, 19, 20, 21, or 22 of not less than 80%, 85%, 90%, 95%, 96%, 97%, 98%, or 99%.
9. The method of any of embodiments 1 to 8,
   a) wherein the vaccine comprises an OspA protein, preferably the heterodimer protein of SEQ ID NO: 1 (Lip-S1D1-S2D1), the heterodimer protein of SEQ ID NO: 2 (Lip-S4D1-S3hybD1) and the heterodimer protein of SEQ ID NO: 3 (Lip-S5D1-S6D1), to be administered
      to a human adult a protein content of said 3 heterodimers in the range of from 120 to 200 μg per dose; or
      to a human child a protein content of said 3 heterodimers in the range of from 60 to 100 μg per dose; or
   b) wherein the vaccine comprises a Clostridium difficile toxin protein, particularly the Clostridium difficile toxin fusion protein of SEQ ID NO: 7 (C-TAB.G5) and/or the Clostridium difficile toxin fusion protein of SEQ ID NO: 8 (C-TAB.G5.1), to be administered to a human at a dose of from 20 to 200 μg.
10. The method of any of embodiments 1 to 9,
   a) wherein the vaccine comprises the heterodimer protein of SEQ ID NO: 1 (Lip-S1D1-S2D1), the heterodimer protein of SEQ ID NO: 2 (Lip-S4D1-S3hybD1) and the heterodimer protein of SEQ ID NO: 3 (Lip-S5D1-S6D1) to be administered
      to a human adult at least three times at a total protein content of said 3 heterodimers in the range of from 120 to 200 μg per dose; or
      to a human child at least three times at a total protein content of said 3 heterodimers in the range of from 60 to 100 μg per dose; or.
   b) wherein the vaccine comprises the Clostridium difficile toxin fusion protein of SEQ ID NO: 7 (C-TAB.G5) and/or the Clostridium difficile toxin fusion protein of SEQ ID NO: 8 (C-TAB.G5.1) to be administered to a human at least three times at a total protein content of said 2 toxin fusion proteins at a dose of from 20 to 200 μg, particularly at a dose of 20 μg at the first administration, at a dose of 75 μg at a second administration and at a dose of 200 μg at a third administration, wherein the second and third administrations are 7 days and 21 days from the first administration, respectively.
11. A method for increasing the bioavailability of a protein in an aluminum-containing vaccine composition, comprising administering the composition to a human, wherein the composition comprises less than 1.25 ppb copper.
12. A method for increasing the bioavailability of a protein in a copper- and aluminum-containing vaccine composition, comprising administering the composition to a human, wherein the composition comprises a sufficient amount of a radical quenching compound.
13. The method of embodiment 11 or 12, wherein the protein is OspA or a Clostridium difficile toxin protein or a SARS-CoV-2 protein or an hMPV protein, preferably an OspA heterodimer or a Clostridium difficile toxin fusion protein or a SARS-CoV-2 spike protein or fragment thereof or an hMPV F protein.
14. The method of any of embodiments 11 to 13, wherein copper is in form of an ion, particularly as Cu⁺ or Cu²⁺.
15. The method of any of embodiments 12 to 14, wherein the radical quenching compound is L-methionine, particularly wherein L-methionine is present in a concentration of at least 10 mmol/l.
16. The method of any of embodiments 11 to 15, wherein the concentration of the radical quenching compound, particularly L-methionine, in mol/l is at least equivalent with the concentration of copper in the composition.
17. The method of any of embodiments 11 to 16, the composition further comprising a reactive compound, wherein the reactive compound is selected from the group consisting of a redox active compound, a radical building compound, a stabilizing compound and a combination of any thereof, especially wherein the reactive compound is selected from the group consisting of formaldehyde, ethanol, chloroform, trichloroethylene, acetone, triton-X-100, triton-X-114, deoxycholate, diethylpyrocarbonate, sulfite, Na₂S₂O₅, beta-propiolactone, polysorbate such as Tween 20®, Tween 80®, O₂, phenol, pluronic type copolymers, and a combination of any thereof.
18. The method of any of embodiments 11 to 17,
   a) wherein the OspA vaccine comprises the heterodimer protein of SEQ ID NO: 1 (Lip-S1D1-S2D1), the heterodimer protein of SEQ ID NO: 2 (Lip-S4D1-S3hybD1) and the heterodimer protein of SEQ ID NO: 3 (Lip-S5D1-S6D1), particularly wherein the composition comprises the heterodimer proteins in a weight ratio of 1:1:1 (Lip-S1D1-S2D1:Lip-S4D1-S3hybD1:Lip-S5D1-S6D1); or
   b) wherein the Clostridium difficile vaccine comprises the Clostridium difficile toxin A protein of SEQ ID NO: 13 (Lip-ToxA-His) and/or the Clostridium difficile toxin B protein of SEQ ID NO: 14 (Lip-ToxB-His), particularly wherein the composition comprises the Clostridium difficile toxin proteins in a weight ratio of 1:1; or
   c) wherein the Clostridium difficile vaccine comprises the Clostridium difficile toxin fusion protein of SEQ ID NO: 7 (C-TAB.G5) and/or the Clostridium difficile toxin fusion protein of SEQ ID NO: 8 (C-TAB. G5.1);
   d) wherein the SARS-CoV-2 vaccine comprises the SARS-CoV-2 spike protein of SEQ ID NO: 15; or
   e) wherein the hMPV vaccine comprises the hMPV F protein of any of SEQ ID NOs: 16-22; or
   f) wherein the vaccine comprises an immunogenic variant of any of the proteins defined in a) to e), particularly wherein the variant has sequence identity to any of the proteins of SEQ ID NOs: 1, 2, 3, 7, 8, 13, 14, 15, 16, 17, 18, 19, 20, 21, or 22 of not less than 80%, 85%, 90%, 95%, 96%, 97%, 98%, or 99%.
19. The method of any of embodiments 11 to 18,
   a) wherein the vaccine comprises a OspA protein, preferably the heterodimer protein of SEQ ID NO: 1 (Lip-S1D1-S2D1), the heterodimer protein of SEQ ID NO: 2 (Lip-S4D1-S3hybD1) and the heterodimer protein of SEQ ID NO: 3 (Lip-S5D1-S6D1), to be administered
      to a human adult a protein content of said 3 heterodimers in the range of from 120 to 200 μg per dose; or
      to a human child a protein content of said 3 heterodimers in the range of from 60 to 100 μg per dose; or
   b) wherein the vaccine comprises a Clostridium difficile toxin protein, particularly the Clostridium difficile toxin fusion protein of SEQ ID NO: 7 (C-TAB.G5) and/or the Clostridium difficile toxin fusion protein of SEQ ID NO: 8 (C-TAB.G5.1), to be administered to a human at a dose of from 20 to 200 μg.
20. The method of any of embodiments 11 to 19,
   a) wherein the vaccine comprises the heterodimer protein of SEQ ID NO: 1 (Lip-S1D1-S2D1), the heterodimer protein of SEQ ID NO: 2 (Lip-S4D1-S3hybD1) and the heterodimer protein of SEQ ID NO: 3 (Lip-S5D1-S6D1) to be administered
      to a human adult at least three times at a total protein content of said 3 heterodimers in the range of from 120 to 200 μg per dose; or
      to a human child at least three times at a total protein content of said 3 heterodimers in the range of from 60 to 100 μg per dose.
   b) wherein the vaccine comprises the Clostridium difficile toxin fusion protein of SEQ ID NO: 7 (C-TAB.G5) and/or the Clostridium difficile toxin fusion protein of SEQ ID NO: 8 (C-TAB.G5.1) to be administered to a human at least three times at a total protein content of said 2 toxin fusion proteins at a dose of from 20 to 200 μg, particularly at a dose of 20 μg at the first administration, at a dose of 75 μg at a second administration and at a dose of 200 μg at a third administration, wherein the second and third administrations are 7 days and 21 days from the first administration, respectively.

The terms “comprising”, “comprise” and “comprises” herein are intended by the inventors to be optionally substitutable with the terms “consisting of”, “consist of” and “consists of”, respectively, in every instance. The term “comprises” means “includes”. Thus, unless the context requires otherwise, the word “comprises”, and variations such as “comprise” and “comprising” will be understood to imply the inclusion of a stated compound or composition (e.g., nucleic acid, polypeptide, antibody) or step, or group of compounds or steps, but not to the exclusion of any other compounds, composition, steps, or groups thereof. The abbreviation, “e.g.” is derived from the Latin exempli gratia, and is used herein to indicate a non-limiting example. Thus, the abbreviation “e.g.” is synonymous with the term “for example”.

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a”, “an”, and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, may be approximate.

The present invention is further illustrated by the following Figures, Tables and Examples, from which further features, embodiments and advantages may be taken. As such, the specific modifications discussed are not to be construed as limitations on the scope of the invention. It will be apparent to the person skilled in the art that various equivalents, changes, and modifications may be made without departing from the scope of the invention, and it is thus to be understood that such equivalent embodiments are to be included herein.

### EXAMPLES

### Example 1. Accelerated Stability Study of an Alum-Adjuvanted Lipidated OspA Heterodimer Antigen

It has previously been observed that high levels of heavy metal contaminants in alum preparations can decrease the stability of antigens in alum-adjuvanted vaccine formulations (see WO2013/083726, which is incorporated herein by reference in its entirety). As the multimeric Borrelia vaccine of the current invention is alum adjuvanted, studies were conducted to determine possible destabilizing effects of heavy metal alum contaminants on vaccine antigens, using the lipidated serotype 5/serotype 6 heterodimer (Lip-S5D1-S6D1 or ST5/ST6; SEQ ID NO: 3) as an example.

Specifically, two alum lots with different levels of residual heavy metal ion impurities were tested side by side in an accelerated stability study of alum-adjuvanted ST5/ST6 heterodimer. Table 1 provides the concentrations of heavy metal ion impurities on the two lots.

TABLE 1
Levels of heavy metal ion impurities in alum lots
A and B used in experimental formulations.

| | | |
|---|---|---|
| Heavy metal | | |
| impurities | Alum lot A | Alum lot B* |

| | | | | |
|---|---|---|---|---|
| Cu | 68 | ng/mL | <25 | ng/mL |
| Fe | 6090 | ng/mL | <4185 | ng/mL |
| Ni | 994 | ng/mL | <179 | ng/mL |

*values below internal quality specifications for all three (ng/mL = ppb; alum is diluted 1:20 in formulations)

Formulations of the Lip-S5D1-S6D1 OspA antigen with and without the excipients L-methionine and sulfite added to the formulation buffer were prepared and placed on accelerated stability at 37° C. The reducing agent sulfite acts as an antioxidant and the amino acid L-methionine acts as a scavenger to protect a protein antigen against oxidation. Table 2 summarizes the formulations.

TABLE 2
Summary of the tested formulations.

| | Lip-S5D1-S6D1 | Alum lot | | |
|---|---|---|---|---|
| | Antigen | (0.5 mg | L-Methionine | Sulfite |
| Formulation | (mg/mL) | Al/mL) | (mM) | (mM) |
| 1 | 0.18 | A | 0 | 0 |
| 3 | 0.18 | A | 10 | 0 |
| 5 | 0.18 | A | 0 | 1 |
| 7 | 0.18 | A | 10 | 1 |
| 9 | 0.18 | B | 0 | 0 |
| 11 | 0.18 | B | 10 | 0 |
| 13 | 0.18 | B | 0 | 1 |
| 15 | 0.18 | B | 10 | 1 |

Formulation buffer: 10 mM PO₄, 150 mM NaCl, 5% Sucrose, pH 6.7, 0.05% Tween 20

Materials and Methods

Materials

Protein OspA Lip-D5B6B, DS 20120904, conc. 1.1 mg/ml

Alum, Brenntag

L-methionine, AppliChem, A 1340

Sodium metabisulfite: Sigma-Aldrich, 13459-500G-R

Sodium dihydrogenphosphate monohydrate: Sigma-Aldrich, S9636

Sodium chloride: VWR, 27810.295

Sucrose: Merck, 1.07653.5000

Tween 20: Merck, 817072.1000

HQ water

Sodium hydroxide solution 1 M (for pH adjustment)

15 ml and 50 ml PP tubes

1.5 ml and 2 ml LoBind tubes

Eppendorf Pipettes and pipette tips

Single use syringes, Omnifix, VWR

Syringe filters Supor, 0.2 μm, Pall

Microfuge 22R, Beckman Coulter

Thermomixer Comfort, Eppendorf

HPLC system Vanquish Flex including a thermostatic column compartment, Thermo RPC-column Zorbax300SB-CN 4.6×150 mm, 5 μm, Agilent, 883995-905

Acetonitrile, VWR 1.00030.2500

Trifluoroacetic acid, Fluka, 91703

Formulation procedure After preparing the formulation buffer (10 mM PO₄, 150 mM NaCl, 5% Sucrose, pH 6.7, 0.05% Tween 20) and the stock solutions of the additives, all three solutions were filtered. The single formulations were prepared in a laminar flow cabinet according to Table 3 by mixing buffer and additives in 15 mL tubes, followed by addition of protein, and mixing and addition of alum. After final mixing, the formulations were aliquoted into 2 mL LoBind tubes and put on accelerated stability at 37° C.

TABLE 3
Pipetting scheme for the preparation of the formulations

| | Lip-S5D1- | Alum | | L-meth | Sulfite | | |
|---|---|---|---|---|---|---|---|
| Formu- | S6D1 | (μl) | | (μl) | (μl) | Buffer | |
| lation | DS (μl) | A | B | 200 mM | 400 mM | (μl) | Total |
| 1 | 667 | 200 | 0 | 0 | 0 | 3133 | 4000 |
| 3 | 667 | 200 | 0 | 200 | 0 | 2933 | 4000 |
| 5 | 667 | 200 | 0 | 0 | 100 | 3033 | 4000 |
| 7 | 667 | 200 | 0 | 200 | 100 | 2833 | 4000 |
| 9 | 667 | 0 | 200 | 0 | 0 | 3133 | 4000 |
| 11 | 667 | 0 | 200 | 200 | 0 | 2933 | 4000 |
| 13 | 667 | 0 | 200 | 0 | 100 | 3033 | 4000 |
| 15 | 667 | 0 | 200 | 200 | 100 | 2833 | 4000 |

Desorption procedure The bound antigen must be desorbed from aluminium hydroxide particles before analysis by RP-HPLC. 250 μl or 450 μl of the thoroughly homogenized DP solution was transferred to 1.5 ml LoBind tubes and centrifuged at 13000×g for 5 minutes. The supernatant was removed. To the remaining pellet, 240 μl or 430 μl, respectively of desorption buffer (500 mM PO₄, pH 7.0+0.05% Tween 20) were added and the pellet was resuspended by pipetting, followed by incubation at 23° C. for 4 hours on a shaker at 750 rpm. After centrifugation (13000×g, 5 minutes) the solution was transferred to a HPLC vial and analyzed by RP-HPLC.

TABLE A-2
RP-HPLC method: Samples were analyzed
by RP-HPLC using a Zorbax300SN-CN column
using the parameters outlined below.

| | |
|---|---|
| Solvents (mobile phase): | Channel A - 0.1% TFA in water |
| | Channel B - 0.1% TFA in acetonitrile |
| Gradient: | 35 → 60% B in 10 min (total run time: 20 min) |
| Gradient details: | 0-2 min: hold at 35% B |
| | 2-12 min: linear gradient 35% to 60% B |
| | 12-13 min: linear gradient 60% to 90% B |
| | 13-13.5 min: hold at 90% B |
| | 13.5-14 min: linear gradient 90% to 35% B |
| | 14-20 min: hold at 35% B (re-equilibration) |
| Flow: | 1 mL/min |
| Column oven | 80° C., including solvent preheating |
| temperature: | |
| Autosampler | 8° C. |
| temperature: | |
| Evaluation: | UV 214 nm |

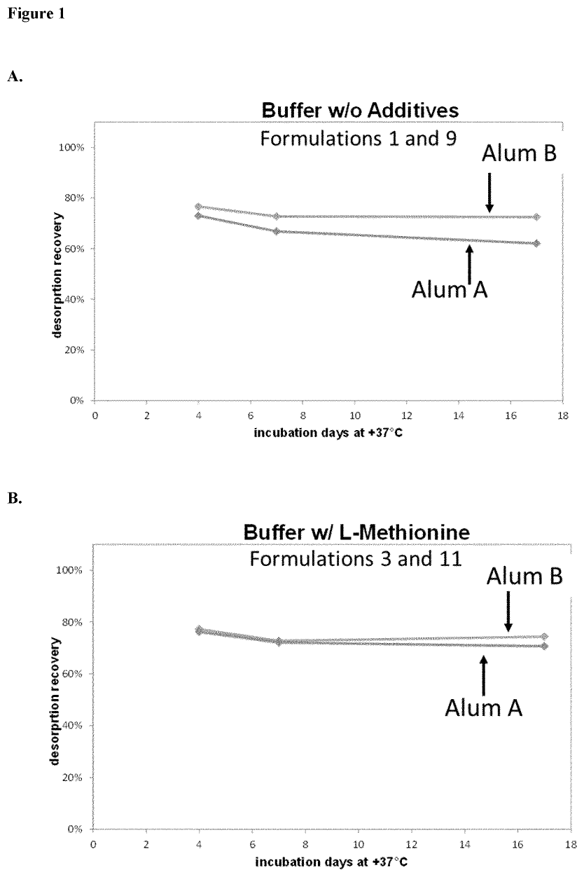

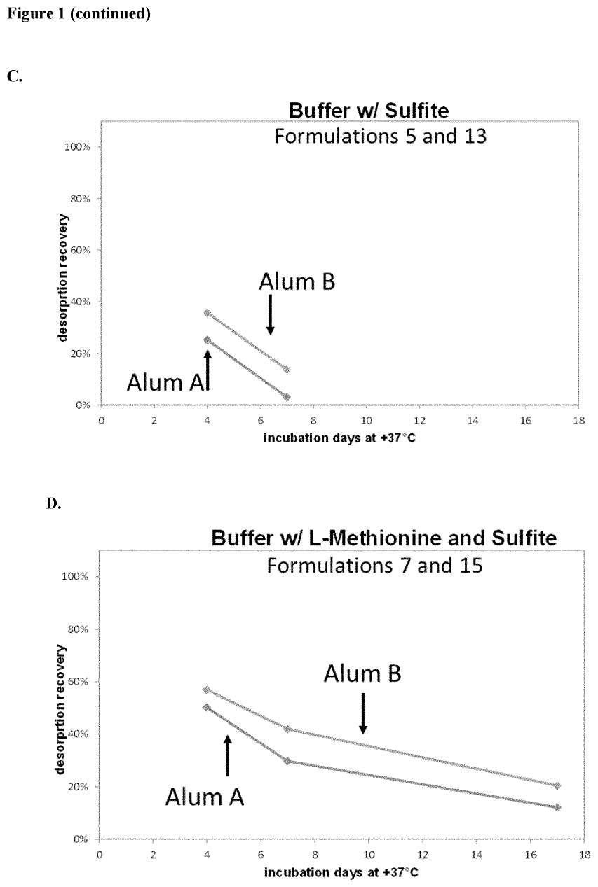

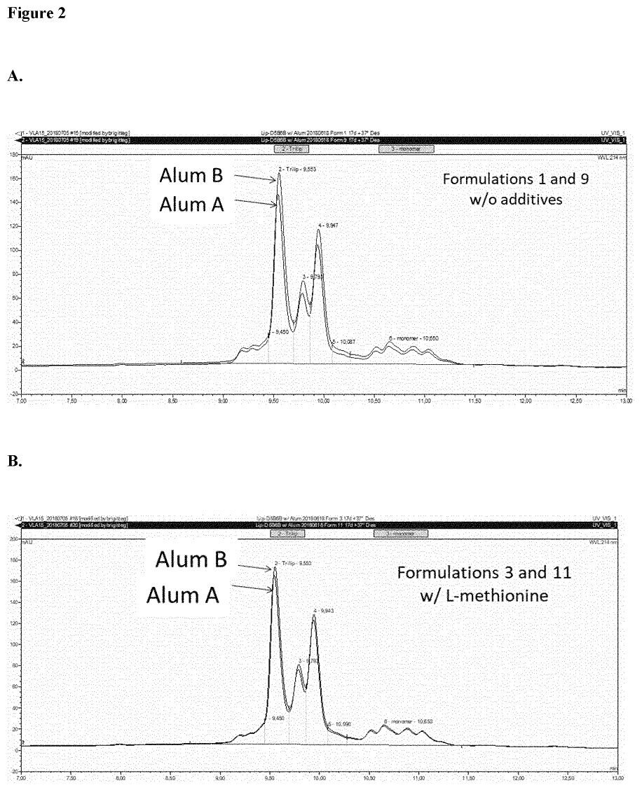

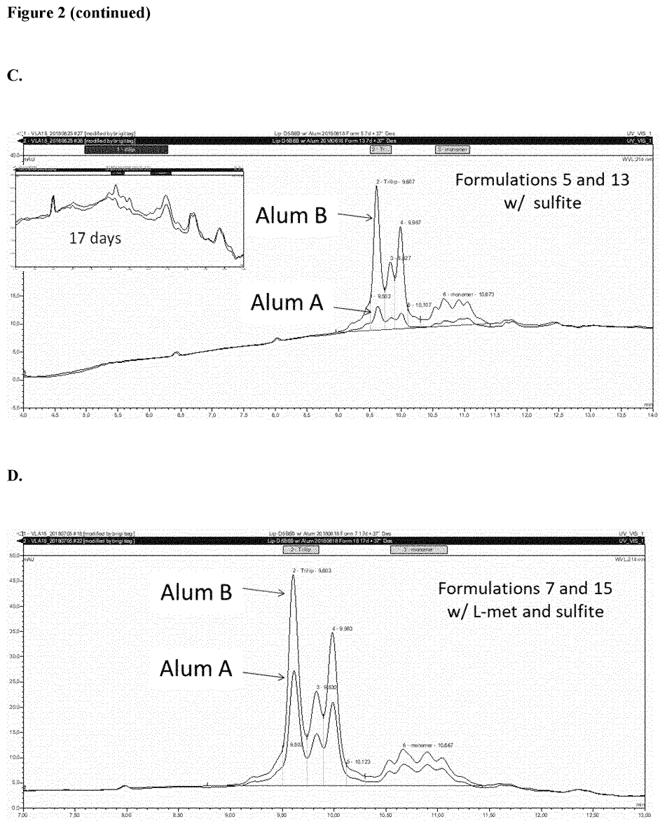

The formulations were stored at +37° C. and samples were taken after 4, 7 and 17 days. The protein was desorbed from the alum and analyzed by RP-HPLC. The results are summarized in Table 4. Samples formulated without excipient (Formulation 1 and 9, FIG. 1A) or formulated with L-methionine (Formulation 3 and 11, FIG. 1B) showed only minor differences between alum A and B. A good stability over time was observed, with the L-methionine containing samples (Formulation 3 and 11) showing a slightly better recovery. The day 17 RP-HPLC overlays for each condition are shown in FIGS. 2A-D.

The samples containing only sulfite as an excipient (Formulation 5 and 13, FIG. 1C) however showed much lower desorption recoveries, with a significant difference between the alum lots. This degrading effect was much more pronounced in formulation 5 containing alum 4230 (A) indicating the negative impact of residual elemental impurities present in alum lot 4230 (A).

L-methionine in presence of sulfite showed a significant stabilizing effect (Formulation 7 and 15, FIG. 1D), compared to formulations containing sulfite only (Formulation 5 and 13). FIGS. 2A-D show RP-HPLC overlays for the four different formulation buffers used, each comparing the 2 different alum lots. Table 4 shows desorption over three time points of the formulations.

In all experiments, complete binding of the antigen on aluminium hydroxide was observed. The lower desorption recovery in formulations containing alum 4230 could be caused by protein modification leading to changes in protein-alum binding strength. Such strong binding can result in reduced immunogenicity (Egan et al., Vaccine 27 (2009) 3175-3180; Hansen et al., Vaccine 27 (2009) 888-892; Hansen et al., Vaccine 25 (2007) 6618-6624; Noe at al., Vaccine 28 (2010) 3588-3594).

TABLE 4
RPC-HPLC results with two alum lots and 4 formulations at three timepoints.

| | | Alum | total desorbed OspA | | purity |
|---|---|---|---|---|---|
| Lip-S5D1-S6D1 w/Alum | | Lot | RPC area | recovery | main peaks |
| Buffer | Form 1 4 d +37° | A | 62.4 | 73% | 76% |
| w/o | Form 1 7 d +37° | | 57.1 | 67% | 75% |
| Additives | Form 1 17 d +37° | | 53.0 | 62% | 72% |
| | Form 9 4 d +37° | B | 65.4 | 77% | 75% |
| | Form 9 7 d +37° | | 62.1 | 73% | 73% |
| | Form 9 17 d +37° | | 62.0 | 73% | 71% |
| Buffer | Form 3 4 d +37° | A | 65.1 | 76% | 76% |
| w/ | Form 3 7 d +37° | | 61.6 | 72% | 76% |
| L-Methionine | Form 3 17 d +37° | | 60.4 | 71% | 74% |
| | Form 11 4 d +37° | B | 66.0 | 77% | 76% |
| | Form 11 7 d +37° | | 62.1 | 73% | 75% |
| | Form 11 17 d +37° | | 63.5 | 74% | 74% |
| Buffer | Form 5 4 d +37° | A | 21.6 | 25% | 71% |
| w/ | Form 5 7 d +37° | | 2.6 | 3% | 54% |
| Sulfite | Form 5 17 d +37° | | no distinct OspA peaks | | |
| | Form 13 4 d +37° | B | 30.5 | 36% | 73% |
| | Form 13 7 d +37° | | 11.7 | 14% | 63% |
| | Form 13 17 d +37° | | no distinct OspA peaks | | |
| Buffer | Form 7 4 d +37° | A | 42.8 | 50% | 74% |
| w/ | Form 7 7 d +37° | | 25.4 | 30% | 71% |
| L-Methionine | Form 7 17 d +37° | | 10.3 | 12% | 63% |
| w/ | Form 15 4 d +37° | B | 48.6 | 57% | 74% |
| Sulfite | Form 15 7 d +37° | | 35.8 | 42% | 72% |
| | Form 15 17 d +37° | | 17.4 | 20% | 67% |

1.-20. (canceled)

21. A method for adjuvanting a vaccine comprising administering an aluminum composition comprising less than 1.25 ppb copper or a sufficient amount of radical quenching compound to a human, wherein the aluminum composition increases the bioavailability of an antigen in the vaccine, wherein the antigen is a protein.

22. A method for vaccinating a human, the method comprising administering an aluminum composition comprising less than 1.25 ppb copper or a sufficient amount of a radical quenching compound to the human, wherein the aluminum composition increases the bioavailability of an antigen in a vaccine, wherein the antigen is a protein.

23. The method of claim 22, wherein the protein is a Borrelia outer surface protein A (OspA) protein or a Clostridium difficile toxin protein or a severe acute respiratory syndrome coronavirus 2 (SARS-CoV-2) protein or a human metapneumovirus (hMPV) protein; preferably an OspA heterodimer protein, a Clostridium difficile toxin fusion protein, a spike protein of SARS-CoV-2, or a fragment thereof or an hMPV fusion (F) protein.

24. The method of claim 22, wherein copper is in form of an ion, particularly as Cu⁺ or Cu²⁺.

25. The method of claim 22, wherein the radical quenching compound is L-methionine, particularly wherein L-methionine is present in a concentration of at least 10 mmol/l.

26. The method of claim 22, wherein the concentration of the radical quenching compound, particularly L-methionine, in mol/l is at least equivalent with the concentration of copper in the composition.

27. The method of claim 22, wherein the composition further comprises a reactive compound, wherein the reactive compound is selected from the group consisting of a redox active compound, a radical building compound, a stabilizing compound and a combination of any thereof, especially wherein the reactive compound is selected from the group consisting of formaldehyde, ethanol, chloroform, trichloroethylene, acetone, triton-X-100, triton-X-114, deoxycholate, diethylpyrocarbonate, sulfite, Na₂S₂O₅, beta-propiolactone, polysorbate such as Tween 20®, Tween 80®, O₂, phenol, pluronic type copolymers, and a combination of any thereof.

28. The method of claim 23,
a) wherein the vaccine comprises the OspA heterodimer protein of SEQ ID NO: 1 (Lip-S1D1-S2D1), the OspA heterodimer protein of SEQ ID NO: 2 (Lip-S4D1-S3hybD1) and the OspA heterodimer protein of SEQ ID NO: 3 (Lip-S5D1-S6D1), particularly wherein the composition comprises the heterodimer proteins in a weight ratio of 1:1:1 (Lip-S1D1-S2D1:Lip-S4D1-S3hybD1:Lip-S5D1-S6D1); or
b) wherein the vaccine comprises a Clostridium difficile toxin A protein of SEQ ID NO: 13 (Lip-ToxA-His) and/or a Clostridium difficile toxin B protein of SEQ ID NO: 14 (Lip-ToxB-His), particularly wherein the composition comprises the Clostridium difficile toxin proteins in a weight ratio of 1:1; or
c) wherein the vaccine comprises a Clostridium difficile toxin fusion protein of SEQ ID NO: 7 (C-TAB.G5) and/or a Clostridium difficile toxin fusion protein of SEQ ID NO: 8 (C-TAB.G5.1); or
d) wherein the vaccine comprises the SARS-CoV-2 spike protein of SEQ ID NO: 15; or
e) wherein the vaccine comprises the hMPV F protein of any one of SEQ ID NOs: 16-22; or
f) wherein the vaccine comprises an immunogenic variant of any one of the proteins defined in a) to e), particularly wherein the variant has sequence identity to any one of the proteins of SEQ ID NOs: 1, 2, 3, 7, 8, 13, 14, 15, 16, 17, 18, 19, 20, 21, or 22 of not less than 80%, 85%, 90%, 95%, 96%, 97%, 98%, or 99%.

29. The method of claim 22,
a) wherein the vaccine comprises an OspA protein, preferably an OspA heterodimer protein of SEQ ID NO: 1 (Lip-S1D1-S2D1), an OspA heterodimer protein of SEQ ID NO: 2 (Lip-S4D1-S3hybD1) and an OspA heterodimer protein of SEQ ID NO: 3 (Lip-S5D1-S6D1), to be administered
to a human adult with a protein content of said 3 heterodimers in the range of from 120 to 200 μg per dose; or
to a human child with a protein content of said 3 heterodimers in the range of from 60 to 100 μg per dose; or
b) wherein the vaccine comprises a Clostridium difficile toxin protein, particularly a Clostridium difficile toxin fusion protein of SEQ ID NO: 7 (C-TAB.G5) and/or a Clostridium difficile toxin fusion protein of SEQ ID NO: 8 (C-TAB.G5.1), to be administered to a human at a dose of from 20 to 200 μg.

30. The method of claim 22,
a) wherein the vaccine comprises an OspA heterodimer protein of SEQ ID NO: 1 (Lip-S1D1-S2D1), an OspA heterodimer protein of SEQ ID NO: 2 (Lip-S4D1-S3hybD1) and an OspA heterodimer protein of SEQ ID NO: 3 (Lip-S5D1-S6D1) to be administered
to a human adult in at least three doses at a total protein content of said 3 heterodimer proteins in the range of from 120 to 200 μg per dose; or
to a human child in at least three doses at a total protein content of said 3 heterodimer proteins in the range of from 60 to 100 μg per dose; or
b) wherein the vaccine comprises a Clostridium difficile toxin fusion protein of SEQ ID NO: 7 (C-TAB.G5) and/or a Clostridium difficile toxin fusion protein of SEQ ID NO: 8 (C-TAB.G5.1) to be administered to a human in at least three doses at a total protein content of said 2 toxin fusion proteins at a dose of from 20 to 200 μg, particularly at a dose of 20 μg at the first administration, at a dose of 75 μg at a second administration and at a dose of 200 μg at a third administration, wherein the second and third administrations are 7 days and 21 days from the first administration, respectively.

31. A method for increasing the bioavailability of a protein in an aluminum-containing vaccine composition, comprising administering the composition to a human, wherein the composition comprises less than 1.25 ppb copper.

32. The method of claim 31, wherein the composition comprises a sufficient amount of a radical quenching compound.

33. The method of claim 31, wherein the protein is a Borrelia outer surface protein A (OspA), a Clostridium difficile toxin protein, a severe acute respiratory syndrome coronavirus 2 (SARS-CoV-2) protein, or a human metapneumovirus (hMPV) protein; preferably an OspA heterodimer, a Clostridium difficile toxin fusion protein, a SARS-CoV-2 spike protein or fragment thereof, or an hMPV fusion (F) protein.

34. The method of claim 31, wherein copper is in form of an ion, particularly as Cu⁺ or Cu²⁺.

35. The method of claim 32, wherein the radical quenching compound is L-methionine, particularly wherein L-methionine is present in a concentration of at least 10 mmol/l.

36. The method of claim 32, wherein the concentration of the radical quenching compound, particularly L-methionine, in mol/l is at least equivalent with the concentration of copper in the composition.

37. The method of claim 31, wherein the composition further comprises a reactive compound, wherein the reactive compound is selected from the group consisting of a redox active compound, a radical building compound, a stabilizing compound and a combination of any thereof, especially wherein the reactive compound is selected from the group consisting of formaldehyde, ethanol, chloroform, trichloroethylene, acetone, triton-X-100, triton-X-114, deoxycholate, diethylpyrocarbonate, sulfite, Na₂S₂O₅, beta-propiolactone, polysorbate such as Tween 20®, Tween 80®, O₂, phenol, pluronic type copolymers, and a combination of any thereof.

38. The method of claim 31,
a) wherein the composition comprises an OspA heterodimer protein of SEQ ID NO: 1 (Lip-S1D1-S2D1), an OspA heterodimer protein of SEQ ID NO: 2 (Lip-S4D1-S3hybD1) and an OspA heterodimer protein of SEQ ID NO: 3 (Lip-S5D1-S6D1), particularly wherein the composition comprises the heterodimer proteins in a weight ratio of 1:1:1 (Lip-S1D1-S2D1:Lip-S4D1-S3hybD1:Lip-S5D1-S6D1); or
b) wherein the composition comprises a Clostridium difficile toxin A protein of SEQ ID NO: 13 (Lip-ToxA-His) and/or a Clostridium difficile toxin B protein of SEQ ID NO: 14 (Lip-ToxB-His), particularly wherein the composition comprises the Clostridium difficile toxin proteins in a weight ratio of 1:1; or
c) wherein the composition comprises a Clostridium difficile toxin fusion protein of SEQ ID NO: 7 (C-TAB.G5) and/or a Clostridium difficile toxin fusion protein of SEQ ID NO: 8 (C-TAB.G5.1);
d) wherein the composition comprises a SARS-CoV-2 spike protein of SEQ ID NO: 15; or
e) wherein the composition comprises a hMPV F protein of any of SEQ ID NOs: 16-22; or
f) wherein the composition comprises an immunogenic variant of any one of the proteins defined in a) to e), particularly wherein the variant has sequence identity to any one of the proteins of SEQ ID NOs: 1, 2, 3, 7, 8, 13, 14, 15, 16, 17, 18, 19, 20, 21, or 22 of not less than 80%, 85%, 90%, 95%, 96%, 97%, 98%, or 99%.

39. The method of claim 31,
a) wherein the composition comprises an OspA protein, preferably an OspA heterodimer protein of SEQ ID NO: 1 (Lip-S1D1-S2D1), an OspA heterodimer protein of SEQ ID NO: 2 (Lip-S4D1-S3hybD1), and an OspA heterodimer protein of SEQ ID NO: 3 (Lip-S5D1-S6D1), to be administered
to a human adult with a protein content of said 3 heterodimers in the range of from 120 to 200 μg per dose; or
to a human child with a protein content of said 3 heterodimers in the range of from 60 to 100 μg per dose; or
b) wherein the composition comprises a Clostridium difficile toxin protein, particularly a Clostridium difficile toxin fusion protein of SEQ ID NO: 7 (C-TAB.G5) and/or a Clostridium difficile toxin fusion protein of SEQ ID NO: 8 (C-TAB.G5.1), to be administered to a human at a dose of from 20 to 200 μg.

40. The method of claim 31,
a) wherein the composition comprises an OspA heterodimer protein of SEQ ID NO: 1 (Lip-S1D1-S2D1), an OspA heterodimer protein of SEQ ID NO: 2 (Lip-S4D1-S3hybD1), and an OspA heterodimer protein of SEQ ID NO: 3 (Lip-S5D1-S6D1) to be administered
to a human adult in at least three doses at a total protein content of said 3 heterodimers in the range of from 120 to 200 μg per dose; or
to a human child in at least three doses at a total protein content of said 3 heterodimers in the range of from 60 to 100 μg per dose.
b) wherein the composition comprises a Clostridium difficile toxin fusion protein of SEQ ID NO: 7 (C-TAB.G5) and/or a Clostridium difficile toxin fusion protein of SEQ ID NO: 8 (C-TAB.G5.1) to be administered to a human in at least three doses at a total protein content of said 2 toxin fusion proteins at a dose of from 20 to 200 μg, particularly at a dose of 20 μg at the first administration, at a dose of 75 μg at a second administration and at a dose of 200 μg at a third administration, wherein the second and third administrations are 7 days and 21 days from the first administration, respectively.
